(19) United States
(12) Patent Application Publication
(10) Pub. No.: US 2023/0025019 A1
(43) Pub. Date: Jan. 26, 2023

(54) VIRTUAL REALITY AND AUGMENTED REALITY HEADSETS FOR MEDITATION APPLICATIONS

(71) Applicant: Sleepme Inc., Mooresville, NC (US)

(72) Inventors: Tara Youngblood, Mooresville, NC (US); Todd Youngblood, Mooresville, NC (US)

(73) Assignee: Sleepme Inc., Mooresville, NC (US)

(21) Appl. No.: 17/870,249

(22) Filed: Jul. 21, 2022

Related U.S. Application Data
(60) Provisional application No. 63/225,183, filed on Jul. 23, 2021.

(51) Int. Cl. A61M 21/02; G16H 20/70; A61B 5/16; A61B 5/024; A61B 5/00; G02B 27/01; G06T 19/00

(52) U.S. Cl. CPC A61M 21/02; G16H 20/70; A61B 5/165; A61B 5/02438; A61B 5/6803; G02B 27/017; G06T 19/006; A61M 2021/0027

(57) ABSTRACT

A virtual reality and augmented reality system configured for meditation applications including a headset comprising an adjustable strap and a display. The headset further includes heating and cooling elements to provide an immersive virtual reality environment to a wearer. The virtual reality system is operable to determine the physiological condition of the wearer and adjust the virtual reality environment based on the physiological condition of the wearer.

### CROSS REFERENCE TO RELATED APPLICATIONS

This application relates to and claims priority from the following applications. This application claims the benefit of U.S. Provisional Patent Application No. 63/225,183, filed Jul. 23, 2021, which is incorporated herein by reference in its entirety.

### BACKGROUND OF THE INVENTION

### 1. Field of the Invention

The present invention relates to virtual reality and augmented reality headsets and more specifically to virtual reality and augmented reality headsets for meditation purposes.

### 2. Description of the Prior Art

It is generally known in the prior art to provide a virtual reality and/or augmented reality headband.

Prior art patent documents include the following:

US Patent Publication No. 2019/0369725 for Guided virtual reality system for relaxing body and mind by inventors Chen et al., filed May 30, 2019 and published Dec. 5, 2019, discloses a guided virtual reality system for relaxing body and mind, including a head-mounted display device, an audio play device, and a video processing device electrically connecting the head-mounted display device and the audio play device. The guided virtual reality system employs a guiding voice and the corresponding virtual scene to guide a user to immerse into the virtual scene. The guiding voice actively changes the virtual scene for the user to deeply experience the sense of immersion, thereby greatly relaxing the body and mind of the user.

US Patent Publication No. 2019/0198153 for System and method for modifying biometric activity using virtual reality therapy by inventors Hill et al., filed Feb. 27, 2019 and published Jun. 27, 2019, discloses systems and methods for using virtual reality content as therapeutic treatment of psychological, psychiatric or medical conditions of a user. The system may comprise a VR device for displaying the VR content to the user and one or more biometric monitors for monitoring the user's biometrics before, during and/or after exposure to the VR content. The system may further include a processor and one or more modules for analyzing the user's biometrics. The method may include the steps of measuring the user's initial biometric data, exposing the user to selected VR content, measuring the user's biometric data during and/or after exposure to the VR content, analyzing changes in the user's biometric data resulting from the selected VR content, determining whether the selected VR content as a positive effect on the psychological, psychiatric or medical condition of the user.

US Patent Publication No. 2020/0276407 for Virtual reality guided meditation in a wellness platform by inventors Goldberg et al., filed Mar. 9, 2020 and published Sep. 3, 2020, discloses a method for providing guided meditation to a user in a virtual reality environment. A user selects a type of meditation, time duration of meditation, and location of meditation. Based on the user's selections, the VR guided meditation system provides a guided meditation exercise and a VR environment to a client device. The guided meditation exercise includes audio instructions guiding the user through meditation steps. The VR environment includes imagery corresponding to the selected location for the meditation exercise, for example, imagery of a beach, waterfall, or trees. The VR guided meditation system also generates reports including statistics of data from a population of users completing guided meditation exercises. For instance, the population of users includes employees of an employer. The report is provided to the employer for the employer to track workplace wellness of the employees.

US Patent Publication No. 2015/0351655 for Adaptive brain training computer system and method by inventor Coleman, filed Jan. 6, 2014 and published Dec. 10, 2015, discloses a computer system for guiding one or more users through a brain state guidance exercise or routine, such as a meditation exercise. The computer system includes at least one computing device which may be a smart phone. A computer program which may be a mobile application runs one or more brain state guidance routines that guide at least one user through at least one brain state guidance exercise. The computing device is connected to at least one bio-signal sensor that provides biofeedback information to the computing device, and where the computer program when executed further measures performance of the at least one user relative to one or more brain state guidance related objectives by analyzing the biofeedback information based on stability of state of mind for the user. The computer program may recognize, score and reward states of meditation.

US Patent Publication No. 2020/0341552 for Feedback device and method for providing thermal feedback by means of same by inventors Yi et al., filed Jul. 9, 2020 and published Oct. 29, 2020, discloses a feedback device and a thermal feedback provision method using the same. The thermal feedback provision method may include checking first operating power applied to a first thermoelectric couple group for a first thermoelectric operation and second operating power applied to a second thermoelectric couple group for a second thermoelectric operation when the first thermoelectric operation is initiated in the first thermoelectric couple group to initiate the output of the first thermal feedback after the second thermoelectric operation is initiated in the second thermoelectric couple group to initiate the output of the second thermal feedback and include applying cognitive enhancement power for enhancing a user's cognition to the first thermoelectric couple group from a time point at which the output of the first thermal feedback is initiated up to a first time point so that the user's cognition of the first thermal feedback is enhanced.

U.S. Pat. No. 10,695,530 for Methods, devices, systems, and kits for regulating skin temperature for mammals to induce and/or maintain sleep by inventor Surace, filed Jun. 10, 2019 and issued Jun. 30, 2020, discloses a subject's forehead, and underlying pre-frontal cortex being cooled by positioning a pre-cooled heat transfer pack on the subject's forehead so that the heat transfer pack is in thermal communication with the subject's forehead, the temperature of the pre-cooled heat transfer pack being below 10° Celsius. Thermal communication between the subject's forehead and the heat transfer pack may be maintained for a period of time sufficient to cool the subject's pre-frontal cortex. Cooling of the pre-frontal cortex may slow the metabolic rate of the subject's pre-frontal cortex and/or induce an onset of sleep for the subject.

US Patent Publication No. 2020/0069966 for Neuro-training device, system and method of use by inventor Porter, filed Oct. 28, 2019 and published Mar. 5, 2020, discloses neuro-training devices, systems and methods of use thereof that utilize encoded light and audio signals, singly or in combination, to stimulate the human brain and synchronize brain wave function. Various embodiments further comprise novel auriculotherapy methods. Embodiments of the neuro-training invention generally comprise a human interface device component(s), an electronic file playback device component, and one or more audio/visual (A/V) files for playback by the playback device component. Light and/or audio signals encoded in the A/V files are run (played) by the playback device component and transmitted to the human interface device component(s). The resulting audio and/or light signals received by the human interface device component(s) and the audio and light transmitted and emitted therefrom and received by the user's eyes and ears maximize neuroplasticity—the brain's ability to reorganize itself by forming new neural connections, resulting in greater brain flexibility and resiliency.

US Patent Publication No. 2020/0345971 for Wearable thermal devices by inventors Schirm et al., filed Apr. 30, 2020 and published Nov. 5, 2020, discloses forehead-mounted temperature regulation apparatuses and methods of using them. These apparatuses may be used for improving sleep. Also included are methods of improving sleep using these apparatuses.

### SUMMARY OF THE INVENTION

The present invention relates to a virtual reality and/or augmented reality headset.

It is an object of this invention to provide a virtual reality and/or augmented reality headband that is configured to create an immersive environment for meditation applications.

In one embodiment, the present invention is directed to a virtual reality system, including a virtual reality headset, including a processor and a memory, configured to fit around a user's head and render virtual environments, at least one headset sensor embedded in the virtual reality headset and/or at least one wearable sensor in communication with the virtual reality headset, at least one thermoelectric module embedded in the virtual reality headset and operable to heat and/or cool a skin-contacting surface of the virtual reality headset, and at least one fluid control unit, in network communication with the virtual reality headset, configured to regulate a temperature of at least one thermoregulating article, wherein the virtual reality headset receives a selection of at least one virtual environment, and wherein the virtual reality headset transmits instructions to the at least one fluid control unit to adjust the temperature of the at least one thermoregulating article based on the selection of the at least one virtual environment.

In another embodiment, the present invention is directed to a virtual reality system, including a virtual reality headset, including a processor and a memory, configured to fit around a user's head and render virtual environments, at least one headset sensor embedded in the virtual reality headset and/or at least one wearable sensor in communication with the virtual reality headset, and a headset control unit, wherein the headset control unit is configured to heat and/or cool fluid, wherein the fluid exits the headset control unit, enters thermal regulation tubes proximate to a skin-contacting surface of the virtual reality headset, and then reenters the headset control unit, wherein the virtual reality headset receives a selection of at least one virtual environment, and wherein the headset control unit heats or cools the fluid based on the at least one selected virtual environment.

In yet another embodiment, the present invention is directed to a virtual reality system, including a virtual reality headset, including a processor and a memory, configured to fit around a user's head and render virtual environments, at least one headset sensor embedded in the virtual reality headset, at least one thermoelectric module embedded in the virtual reality headset and operable to heat and/or cool a skin-contacting surface of the virtual reality headset, and wherein the at least one headset sensor includes at least one heart rate variability sensor and/or at least one electroencephalography (EEG) sensor, wherein the at least one headset sensor generates sensor data indicating a stress level of a user, wherein the virtual reality headset receives a selection of at least one virtual environment, and wherein the at least one virtual environment is automatically changed based on the sensor data, generated by the at least one headset sensor.

These and other aspects of the present invention will become apparent to those skilled in the art after a reading of the following description of the preferred embodiment when considered with the drawings, as they support the claimed invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates a virtual reality headset according to one embodiment of the present invention.

FIG. 2 illustrates a virtual reality system according to one embodiment of the present invention.

FIG. 3 illustrates a rear exploded perspective of a virtual reality headset according to one embodiment of the present invention.

FIG. 4 illustrates a front perspective view of a headset according to one embodiment of the present invention.

FIG. 5 illustrates a control unit according to one embodiment of the present invention.

FIG. 6 illustrates a side exploded perspective view according to one embodiment of the present invention.

FIG. 7 illustrates a control unit according to one embodiment of the present invention.

FIG. 8 illustrates a schematic diagram of a virtual reality system according to one embodiment of the present invention.

FIG. 9 illustrates a schematic diagram of a virtual reality system according to one embodiment of the present invention.

FIG. 10 illustrates a virtual reality environment according to one embodiment of the present invention.

FIG. 11 illustrates a virtual reality environment according to one embodiment of the present invention.

FIG. 12 illustrates a virtual reality environment according to one embodiment of the present invention.

FIG. 13 illustrates a virtual reality environment according to one embodiment of the present invention.

FIG. 14 illustrates a virtual reality environment according to one embodiment of the present invention.

FIG. 15 illustrates a virtual reality environment according to one embodiment of the present invention.

FIG. 16 illustrates a virtual reality environment according to one embodiment of the present invention.

FIG. 17 illustrates a schematic diagram according to one embodiment of the present invention.

### DETAILED DESCRIPTION

The present invention is generally directed to virtual reality and/or augmented reality headbands that are configured to support meditation applications.

In one embodiment, the present invention is directed to a virtual reality system, including a virtual reality headset, including a processor and a memory, configured to fit around a user's head and render virtual environments, at least one headset sensor embedded in the virtual reality headset and/or at least one wearable sensor in communication with the virtual reality headset, at least one thermoelectric module embedded in the virtual reality headset and operable to heat and/or cool a skin-contacting surface of the virtual reality headset, and at least one fluid control unit, in network communication with the virtual reality headset, configured to regulate a temperature of at least one thermoregulating article, wherein the virtual reality headset receives a selection of at least one virtual environment, and wherein the virtual reality headset transmits instructions to the at least one fluid control unit to adjust the temperature of the at least one thermoregulating article based on the selection of the at least one virtual environment.

In another embodiment, the present invention is directed to a virtual reality system, including a virtual reality headset, including a processor and a memory, configured to fit around a user's head and render virtual environments, at least one headset sensor embedded in the virtual reality headset and/or at least one wearable sensor in communication with the virtual reality headset, and a headset control unit, wherein the headset control unit is configured to heat and/or cool fluid, wherein the fluid exits the headset control unit, enters thermal regulation tubes proximate to a skin-contacting surface of the virtual reality headset, and then reenters the headset control unit, wherein the virtual reality headset receives a selection of at least one virtual environment, and wherein the headset control unit heats or cools the fluid based on the at least one selected virtual environment.

In yet another embodiment, the present invention is directed to a virtual reality system, including a virtual reality headset, including a processor and a memory, configured to fit around a user's head and render virtual environments, at least one headset sensor embedded in the virtual reality headset, at least one thermoelectric module embedded in the virtual reality headset and operable to heat and/or cool a skin-contacting surface of the virtual reality headset, and wherein the at least one headset sensor includes at least one heart rate variability sensor and/or at least one electroencephalography (EEG) sensor, wherein the at least one headset sensor generates sensor data indicating a stress level of a user, wherein the virtual reality headset receives a selection of at least one virtual environment, and wherein the at least one virtual environment is automatically changed based on the sensor data, generated by the at least one headset sensor.

None of the prior art discloses virtual reality and augmented reality headsets that include temperature control and feedback elements and are operable for meditation applications. Furthermore, prior art fails to include a virtual reality and augmented reality system that includes a feedback loop to moderate and influence cooling programs based on brain activity.

Many people suffer from anxiety, stress, depression, and other mental health conditions. These mental health conditions often affect the sleep and day-to-day performance of an individual. One method to help with these mental health conditions is meditation. Generally, meditation requires an individual to get comfortable, to focus on their breath and how their body moves with each inhale and exhale. This is challenging for individuals with busy daily schedules and/or noisy and crowded environments. Furthermore, many individuals need to be trained on how to meditate and require a teacher and/or step by step guidance on how to meditate. Currently, this typically requires attending in-person meditation classes or having accessing to a remote device, which is difficult to watch and hear in a noisy environment. One way to overcome this problem is to create an immersive virtual reality environment that provides audiovisual guidance so a wearer is able to successfully meditate. Therefore, there is a need for a virtual reality system that provides an immersive environment for meditation applications even in noisy environments.

Sometimes audiovisual cues are not enough to provide a completely immersive environment. To amplify the virtual reality environment, a feedback element is needed. Temperature related feedback elements are particularly beneficial for creating sensations for a wearer. Additionally, monitoring a wearer's temperature and creating a response based on the wearer's temperature improves the overall effectiveness of a virtual reality/augmented reality system. Therefore, there is a need for a virtual reality system that is operable to create an immersive environment using temperature related elements to provide feedback to a wearer.

The present invention includes a virtual reality system with temperature controlling elements to create an immersive environment to provide a relaxing and mediative environment. The virtual reality system includes a virtual reality headset that is operable to display virtual reality media including virtual reality images, scenes, movies and/or environments. The virtual reality system is further operable to change the displayed media based on the physiological condition of the wearer. Advantageously, this enables the present invention to adapt to a plurality of wearers and provide a unique immersive virtual reality environment for each wearer.

The virtual reality system is configured to block out external light and display an audiovisual representation to a wearer. The virtual reality system is configured to change the display based on the wearer's viewing direction and provide a full 360 degree virtual environment. Advantageously, the virtual reality system is configured to only play ether audio or visual data based on a wearer's preference.

Referring now to the drawings in general, the illustrations are for the purpose of describing one or more preferred embodiments of the invention and are not intended to limit the invention thereto.

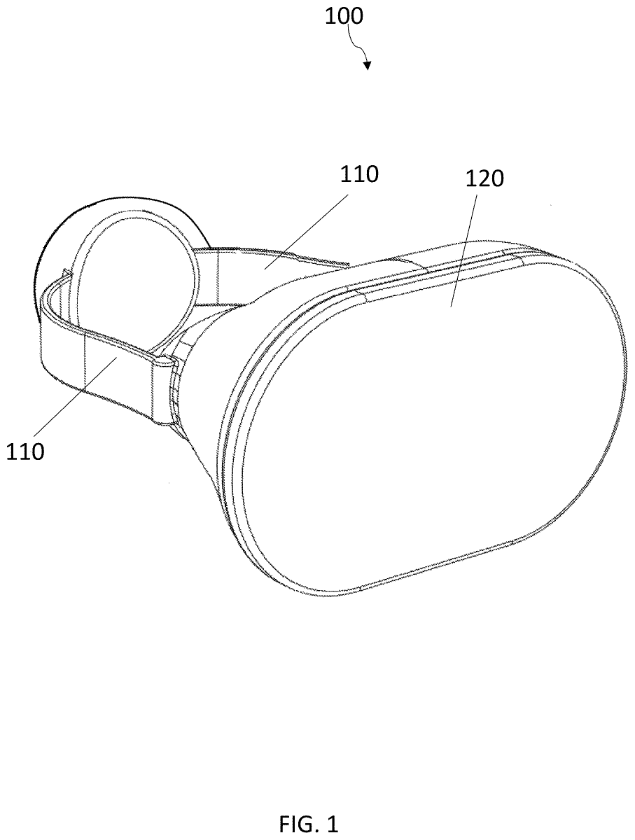

FIG. 1 illustrates a headset according to one embodiment of the present invention. The headset 100 includes a virtual reality display, at least one strap 110, an eyewear component 120, at least one power supply component, at least one sensor, at least one image capturing device, and control electronics. In one embodiment, the power supply component includes a battery, a supercapacitor, or other similar power supply components. In another embodiment, the battery includes a rechargeable battery. In yet another embodiment, the rechargeable battery includes a lithium ion battery. In one embodiment, the virtual reality system includes at least one temperature assembly. The temperature assembly is configured to heat or cool a wearer based on a desired effect.

The headset is configured for virtual reality, augmented reality and/or mixed reality environments. The headset is configured to receive and display an image of a virtual scene, movie, and/or environment. The headset is further operable to receive audio data and communicate the audio data to a wearer via a speaker, headphones, and other similar audio playback devices. For example, and not limitation, the headset is configured to receive audiovisual data including a meditation class set at the beach. The headset is configured to display the beach environment via the display and play corresponding sounds as well as the voice of a teacher leading the meditation class via the audio playback device. In one embodiment, the headphones are noise-cancelling headphones. The noise-cancelling headphones are configured to block out external noise so a wearer is completely immersed in the virtual reality environment.

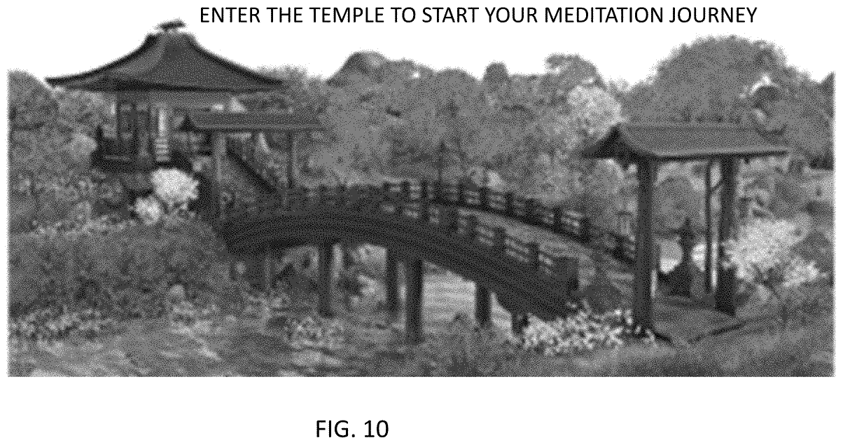

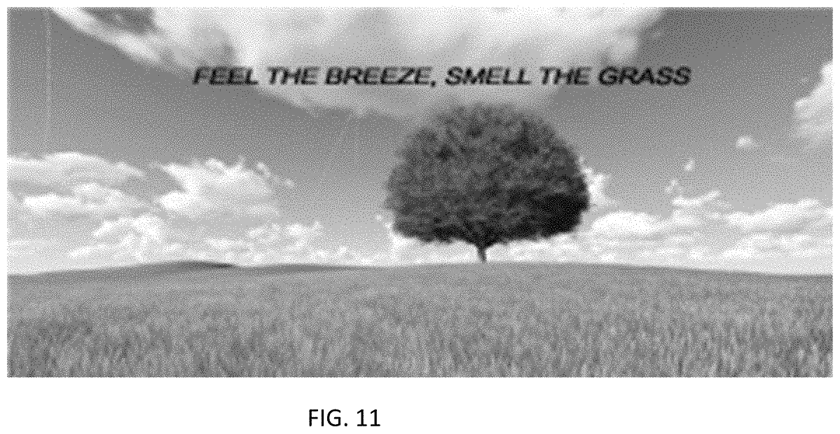

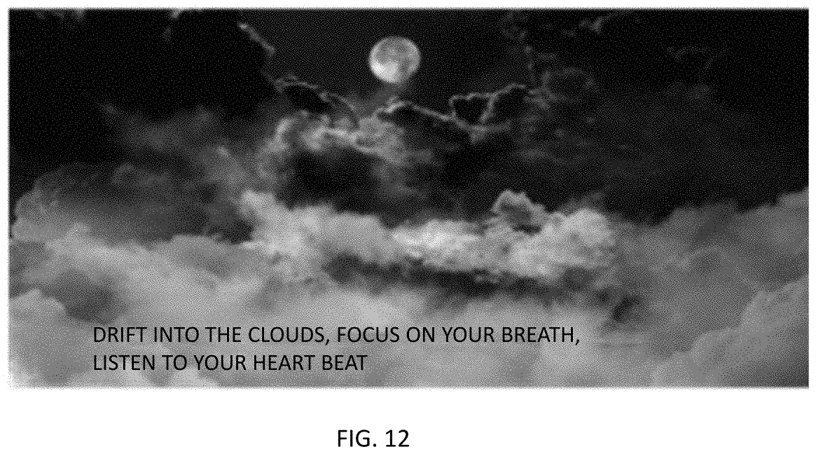

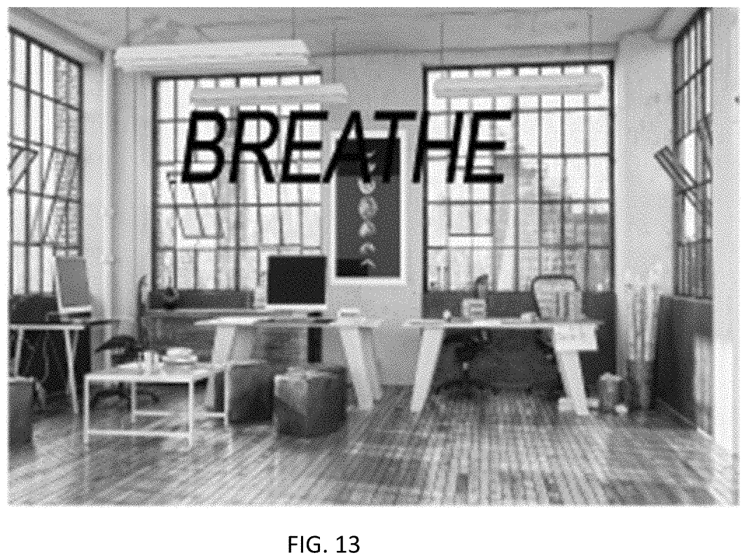

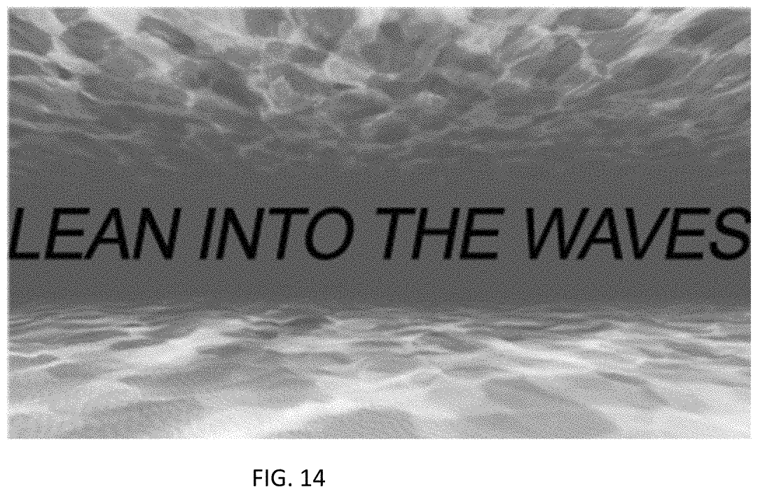

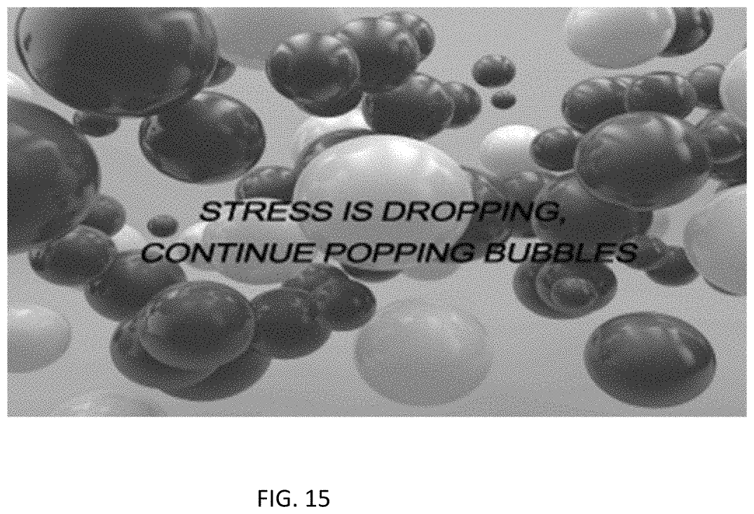

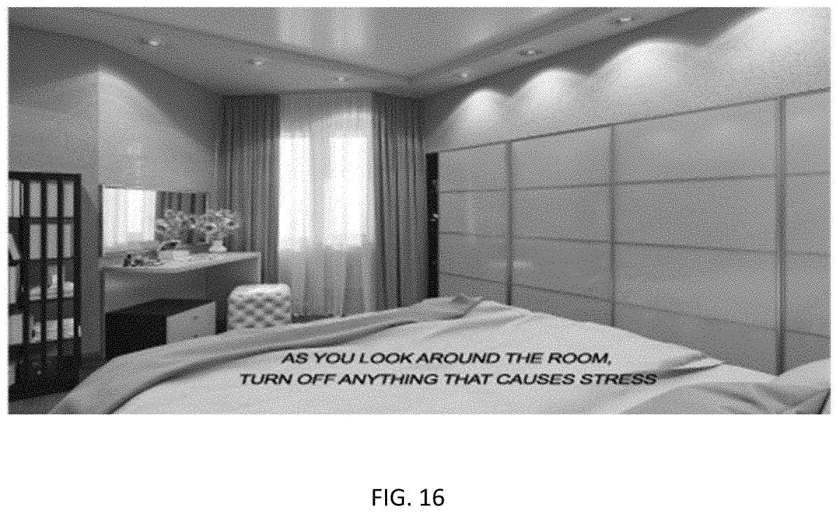

The virtual reality system is configured to provide a meditation exercise and a corresponding virtual reality environment based on a wearer's input and/or preferences. The meditation exercise includes audio instructions guiding the wearer through meditation steps. The virtual reality environment includes imagery corresponding to the selected location for the meditation exercise. The meditation imagery includes a beach, waterfall, trees, mountain range, lakes, large landscapes, and other desired environments. Other examples of meditation imagery include the virtual reality environments shown in FIGS. 10-16.

In one embodiment, the at least one strap is configured to wrap around a wearer's head and attach to the eyewear component 120 via at least one attachment mechanism. The at least one attachment mechanism includes a hook and loop fastener, a latch, a button and other similar attachment mechanisms. The at least one strap is adjustable to a wearer's head. Advantageously, this allows the headset to be used for wearers of different head sizes. For example, and not limitation, the at least one strap includes a tightening mechanism. The tightening mechanism is configured to rotate in one direction and increase the tension in the head strap and rotate in the opposite direction to loosen the tension in the head strap. In yet another embodiment, the at least one strap includes at least two straps. In one embodiment, the at least two straps do not overlap and are in a parallel position around a wearer's head. Alternatively, the at least two straps are configured to intersect in the center of the back of a wearer's head to provide a tighter fit.

In yet another embodiment, the present invention includes a cap or helmet. Alternatively, the present invention includes a pillow or a chair. The cap or helmet includes at least one snap or at least one clip. In another embodiment, the present invention includes a single piece headgear that is configured to expand as a wearer presses the headgear on the wearer's head. In another embodiment, the headgear includes a material. The material includes at least one of the following: honeycomb plastic, spacer fabrics, nylon, polystyrene, carbon fiber, and other similar materials.

Advantageously, the headset is configured to provide minimal pressure to a wearer's face. The headset is designed so a wearer comfortably rests their nose inside a nose compartment. In one embodiment, the nose component is adjustable. The nose component is configured to move left, right, up, and/or down, and is operable to expand. In another embodiment, the nose compartment includes temperature feedback elements to help a wearer's breathing and to provide temperature feedback. For example, and not limitation, the headset is configured to determine when a wearer is having problems breathing and/or a sinus problem. The headset is configured to apply heat to a wearer's sinus area to relieve pressure. Alternatively, the headset is designed to rest on the ridge of the wearer's nose. In yet another embodiment, the headset covers a wearer's entire face.

In one embodiment, the at least one image capturing device is a motion sensor camera and is configured to capture a wearer's body movement. Alternatively, the image capturing device is a LIDAR camera. The virtual reality system is configured to use the captured motion data to determine a wearer's state of mind and whether the displayed virtual reality imagery needs to be changed. For example, if the motion capture data indicates that a wearer is restless then the virtual reality system is configured to display a peaceful scene (e.g. a calm day at the beach) and a corresponding calming noise (e.g. waves crashing). The at least one image capturing device is further operable to determine a wearer's posture and provide at least one recommendation to correct a wearer's posture based on the audiovisual display. The posture feedback is critical for meditation applications to ensure a wearer is in a proper meditation position.

In another embodiment, the virtual reality system is operable to capture the wearer's brain waves and heart rate variability via the at least one sensor (e.g. electroencephalogram (EEG) sensor). The virtual reality system is configured to instruct a level of breathing and intervention based on the brain waves and heart rate variability. In one embodiment, the intervention includes suggesting a yoga position such as yoga nidra or yogic sleep. Alternatively, the present invention is operable to suggest stretching, walking, or changing locations. The virtual reality system further includes an algorithm to adjust to a wearer's natural temperature fluctuations and includes a plurality of temperature settings. Advantageously, the present invention is configured to lower sleep latency, lower insomnia severity, and improve sleep quality.

The control electronics preferably have at least one processor. By way of example, and not limitation, the processor includes a general-purpose microprocessor (e.g., a central processing unit (CPU)), a graphics processing unit (GPU), a microcontroller, a Digital Signal Processor (DSP), an Application Specific Integrated Circuit (ASIC), a Field Programmable Gate Array (FPGA), a Programmable Logic Device (PLD), a controller, a state machine, gated or transistor logic, discrete hardware components, or any other suitable entity or combinations thereof that can perform calculations, process instructions for execution, and/or other manipulations of information. In one embodiment, one or more of the at least one processor is operable to run predefined programs stored in at least one memory of the control electronics.

The control electronics preferably includes at least one antenna, which allows the control electronics to receive and process input data (e.g., temperature settings, virtual reality settings) from at least one remote device (e.g., smartphone, tablet, laptop computer, desktop computer, remote control). In a preferred embodiment, the at least one remote device is in wireless network communication with the control electronics. The wireless communication is, by way of example and not limitation, radiofrequency, BLUETOOTH®, ZIGBEE®, WI-FI®, wireless local area networking, near field communication (NFC), or other similar commercially utilized standards. Alternatively, the at least one remote device is in wired communication with the control electronics through USB or equivalent.

In one embodiment, the at least one processor is a microcontroller. The microcontroller includes a transceiver, BLUETOOTH module, WI-FI module, a microprocessor, an ultra-low-power co-processor, read-only memory (ROM), random-access memory (RAM) (e.g., static random-access memory (SRAM)), flash memory, a power management unit, a temperature sensor, and/or a digital-to-analog converter.

In yet another embodiment, the virtual reality system includes a temperature component configured to provide a heating or cooling effect to a wearer. In one embodiment, the headset includes waterproof tubing and a fluid reservoir. The temperature component includes a thermoelectric device that is configured to heat or cool the fluid reservoir. The headset is configured to heat or cool the fluid reservoir based on the wearer's needs or preferences and then move the fluid through the tubing to provide a cooling or heating effect to the wearer.

In another embodiment, the headset includes at least one fluid reservoir holding fluid to be circulated through the headset. The at least one fluid reservoir is positioned adjacent to at least one temperature component. The at least one temperature component includes at least one thermoelectric module containing at least one Peltier chip. In one embodiment, the at least one thermoelectric module contains four Peltier chips. When electricity is provided to the at least one thermoelectric module, the at least one Peltier chip causes heating or cooling of the at least one fluid reservoir. The at least one thermoelectric module is connected to a at least one heat sink via at least one heat tube. In one embodiment, the at least one heat sink includes a first heat sink and a second heat sink that are aligned along a common axis with space separating the first heat sink from the second heat sink. The at least one thermoelectric module is positioned such that it is not aligned along the common axis of the first heat sink and the second heat sink and such that the at least one thermoelectric module is not located in the space between the first heat sink and the second heat sink. When the at least one thermoelectric module is in cooling mode, heat is transferred away from the at least one thermoelectric module and toward the first heat sink and the second heat sink via the at least one heat tube. This transfer of heat increases the efficiency of the at least one thermoelectric module and prevents the at least one thermoelectric module from breaking down due to excessive heat buildup.

In one embodiment, the first heat sink and the second heat sink each include a plurality of fins, with the fins assisting in dissipating excess heat. The plurality of fins each extend perpendicularly outward from a base of each of the first heat sink and second heat sink. In one embodiment, the first heat sink is connected to at least one first fan and the second heat sink is connected to at least one second fan. When active, at least one first fan blows air over the first heat sink in a first direction and the at least one second fan sucks air from the second heat sink in the first direction, thereby creating a common air path along the common axis between the first heat sink and the second heat sink. The common air path increases the efficiency of the first heat sink and the second heat sink. Additionally, because the at least one thermoelectric module is positioned such that it is not aligned along the common axis of the first heat sink and the second heat sink, and is therefore not in the common air path, air possessing residual heat from the first heat sink and the second heat sink is not blown directly over the at least one thermoelectric module.

In one embodiment, the first heat sink and the second heat sink are connected by a chassis, which acts as an electromagnetic field (EMF) shield. In one embodiment, the chassis is a strip having approximately the same thickness as the first heat sink and second heat sink. In another embodiment, the chassis substantially surrounds the interior of the control electronics. In one embodiment, the chassis is formed from one or more sheet metals, such as copper, aluminum, cobalt, brass, nickel, silver, steel, tin, or other metals able to act as a shield to EMF radiation. In another embodiment, the chassis is formed from a metal foam or from a conductive non-metal fabric capable of stopping EMF radiation, such as nylon coated or interwoven with a conductive metal material.

EMF radiation has been shown, in some cases, to increase the quantity of melatonin produced during sleep, which potentially causes a poorer quality of sleep. In addition, individuals have been shown to experience symptoms including headaches, nausea, and fatigue when exposed to EMF radiation, especially over longer periods of time. Therefore, the inclusion of the chassis in the control electronics helps prevent prolonged exposure to wearers of EMF radiation produced by the control electronics operation, which otherwise disrupts a wearer's meditation, sleep or cause other adverse symptoms.

Alternatively, the headset includes a plurality of thermoelectric temperature regulators arranged across the virtual reality headset, a controller configured to apply power to cool the thermoelectric temperature regulators to between about 1° C. and about 30° C., and a holding mechanism configured to secure the headset to the wearer's face and/or forehead. In one embodiment, the controller is configured to control the applied temperature during an active cooling phase for a predetermined time period (e.g., 10 minutes to 60 minutes, 10 minute to 45 minutes, etc. or any time between 10 minutes and 3 hours, selected by the wearer via a remote device or the headset or automatically determined), or until the headset detects that the wearer is asleep. The headset is then operable to toggle into a standby state and regulate the temperature at a standby temperature (e.g., between about 26° C. and about 38° C., between about 28° C. and about 38° C., between 30° C. and 38° C., etc. or within a few degree, e.g., +/−2° C., of body surface/skin temperature). In some variations, the headset is configured to apply the cooling in a ramp with decreasing temperature (e.g., decreasing from 30° C. or 25° C.) until the wearer experiences a diving reflex, at which point the temperature is sustained for a predetermined first treatment time period (e.g., 10 minutes to 60 minutes, 10 minute to 45 minutes, etc. or any time between 10 minutes and 3 hours).

In another embodiment, the controller is configured to apply energy in a pulsatile manner so that the thermoelectric temperature regulators apply variable cooling to the wearer's face (e.g., varying between the cooling temperature and a temperature that is slightly higher than the cooling temperature). For example, the controller is configured to apply the energy in pulses having a pulse duration of less than 360 seconds.

An example of a cooling headband is disclosed in U.S. Patent No. 2017/0333667, which is incorporated herein by reference in its entirety.

In yet another embodiment, the virtual reality system is operable to receive wearer voice input data. The virtual reality system includes a microphone that is operable to receive and record a wearer's voice. The headset is further operable to change the audiovisual display and/or the temperature component based on the wearer's words. For example, and not limitation, the virtual reality system is configured to receive the words “Too Hot” from a wearer, and activate the cooling element of the temperature component.

Additionally, the virtual reality system further comprises a sensory feedback device such as a lamp, which is electrically wired or connected to the headset. The lamp is operable to provide haptic feedback based on the audiovisual display. For example, and not limitation, the lamp is configured to generate a light on a wearer to simulate sunlight and heat. In another embodiment, the virtual reality system includes an actuator. The virtual reality system is configured to activate the actuator to provide haptic feedback based on wearer data captured by at least one sensor. For example, and not limitation, the virtual reality system is operable to determine that a wearer has poor posture during a mediation exercise and then activate the actuator until the wearer has good posture. In yet another embodiment, the virtual reality system is configured to provide temperature feedback based on the wearer's data. For example, and not limitation, the virtual reality system is configured to generate a cooling effect when the virtual reality system detects that a wearer is sweating, overheating, experiencing an elevated heart rate, increased stress levels and/or other similar symptoms.

In one embodiment, the headset is further configured to work with a sleep and promotion system as disclosed in US Patent Publication No. 20180110960, which is incorporated herein by reference in its entirety.

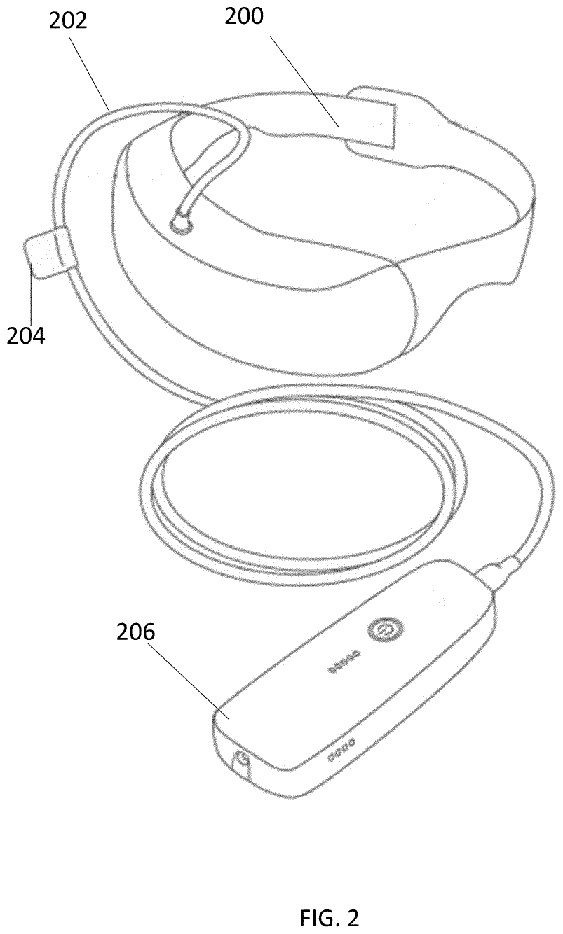

FIG. 2 illustrates a virtual reality system according to one embodiment of the present invention. The virtual reality system includes a headset 200, a power cable 202, a wire management tab 204, and a control unit 206. The headset 200 includes an adjustable strap to conform to a wearer's head. The headset is connected to the control unit 206 via the power cable 202. The power cable 202 includes the wire management tab 204 to prevent the power cable from interfering with the wearer's vision and/or movement. The control unit is configured to power the headset on and off, and control the temperature of the headset. Advantageously, the control unit is configured to receive a power supply cable and is configured to charge the headset. In one embodiment, the headset is configured to function after receiving a command from the control unit and the control unit is no longer attached via the power cable.

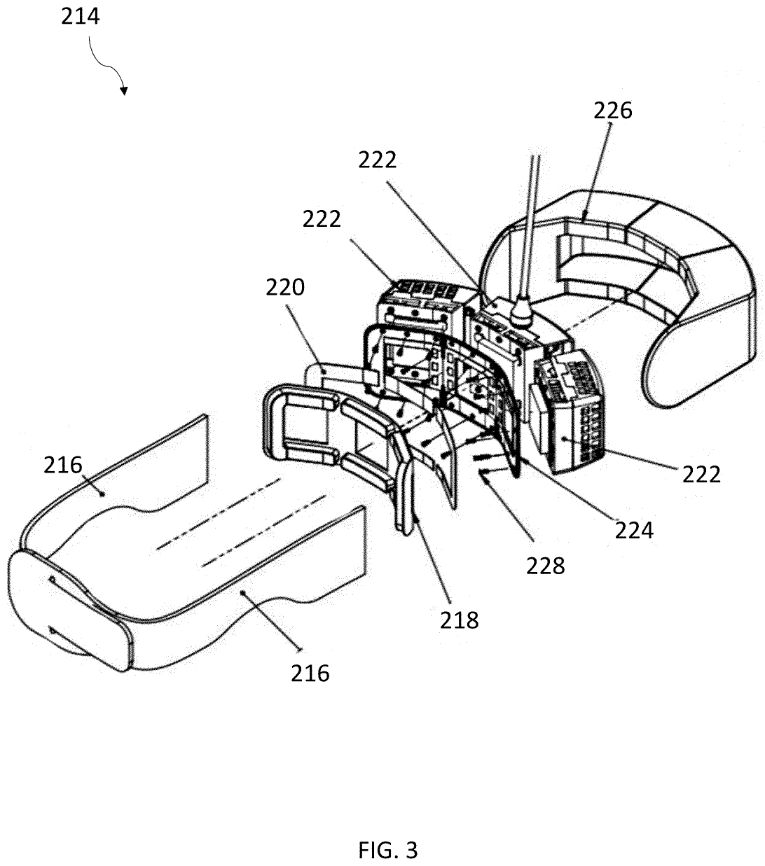

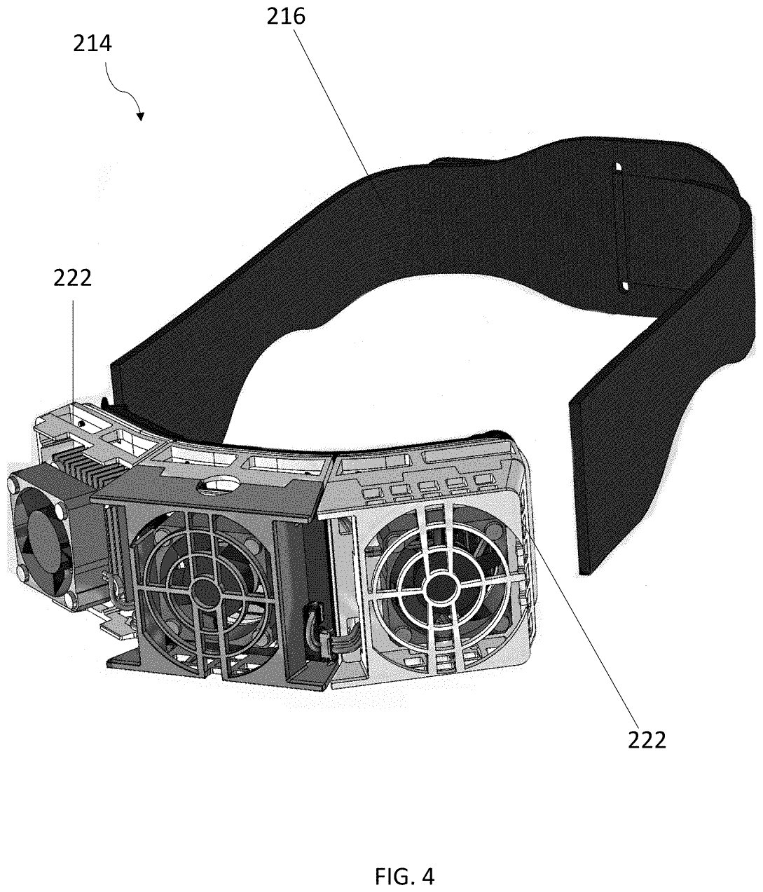

FIG. 3 illustrates an exploded view of a headset according to one embodiment of the present invention. The headset 214 includes a fabric strap 216, a comfort pad assembly 218, an adhesive loop 220, a cooling engine assembly 222, a hinge plate 224, a cover 226, and at least one screw 228. FIG. 4 illustrates a front perspective view of the headset according to one embodiment of the present invention.

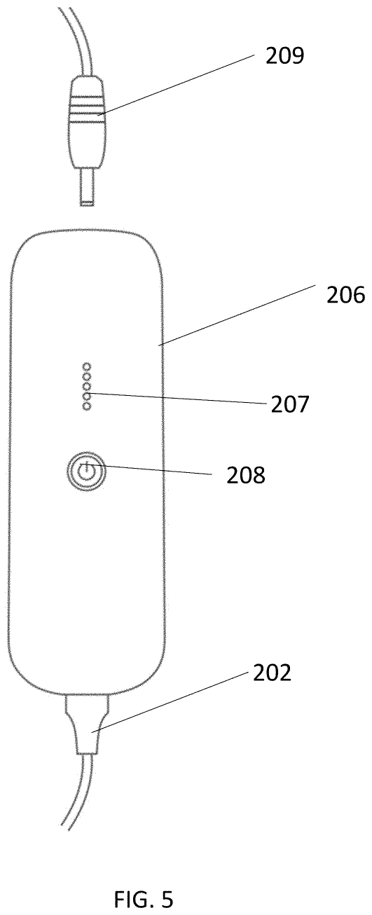

FIG. 5 illustrates a top view of a control unit according to one embodiment. The control unit 206 includes a power output cable 202, a power input cable 209, a visual indicator 207, and a power button 208. The control unit 206 is configured to receive power via the power input cable 209 from an external power source. The control unit 206 is configured to provide power to a headset via the power output cable 202. The control unit is further operable to power on and power off the headset via the power button 208. In one embodiment, the visual indicator 207 includes at least one light-emitting diode (LED) indicator. In another embodiment, the visual indicator includes five LED indicators. The control unit is configured to activate a number of LED indicators corresponding to the battery level of the headset. For example, and not limitation, the control unit is configured to activate five LED indicators when the headset is fully charged and one LED indicator when the headset is low on battery. Alternatively, the visual indicator corresponds to the temperature level of the headset.

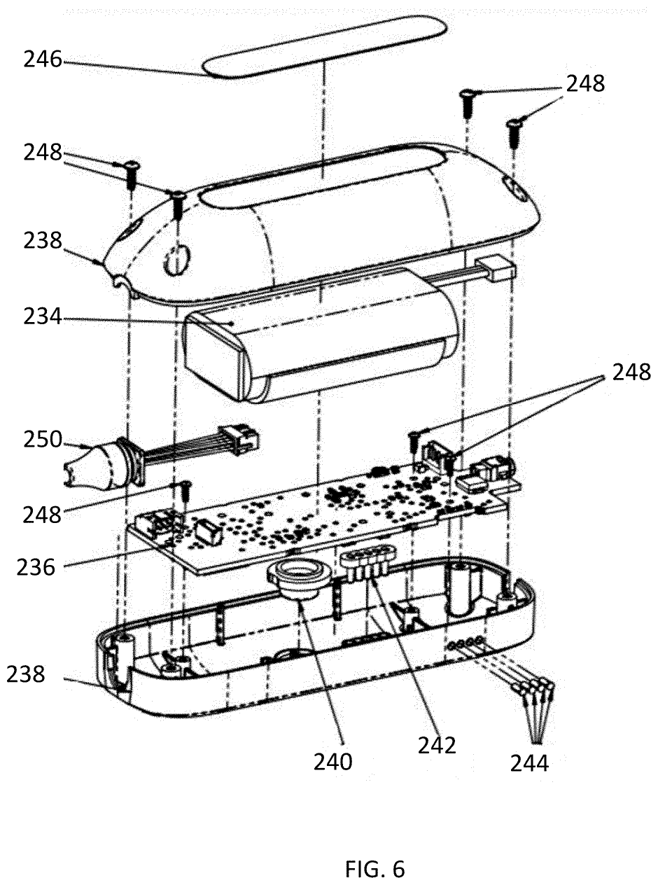

FIG. 6 illustrates an exploded view of a control unit according to one embodiment of the present invention. The control unit includes a battery assembly 234, a printed circuit board assembly 236, a housing 238, a power button 240, a temperature light pipe 242, a battery light pipe 244, a label 246, at least one screw 248, and a cable 250. In one embodiment, the control unit includes a LED display to indicate the temperature level. For example, and not limitation, the LED display includes at least 5 LED lights and is configured to illuminate a light based on the temperature level. One activated light is the lowest setting and five activated lights is the highest setting.

In one embodiment, the headset includes an AC power input voltage between about 100V to about 240V with about 50-60 Hz and about 3 A. In yet another embodiment, the headset includes a DC power of 12 VDC and 1 Mbps. In another embodiment, the headset is operable to function between about 14° C. to about 24° C. (57° F. to 75° F.) and about 15% to about 75% relative humidity, non-condensing.

In another embodiment, the headset includes control electronics configured to determine if the device is too cold and/or too warm and is configured to shut down the headset based on the temperature of the headset. The control electronics is further configured to monitor current through the headset. In one embodiment, the headset includes cooling engines as well as thermistors. The headset includes at least one sensor configured to measure the cold side temperature of each thermistor. The headset further includes a fan and thermoelectric cooling (TEC) current sensors, and is configured to monitor battery voltage and battery temperature. In yet another embodiment, two cooling engines are made of a TEC sensor mounted to a heatsink. The fan is configured to dissipate heat from the cooling engines. In another embodiment, the present invention is sealed with room temperature vulcanizing (RTV) to prevent moisture condensation on the temperature elements. In yet another embodiment, two thermoelectric coolers are wired in parallel. In one embodiment, the virtual reality system includes a heat sink including a black anodized extruded aluminum sink that is configured to dissipate heat from the thermoelectric cooler. The present invention further includes a thermal tape to attach the thermoelectric cooler to the heatsink and transfer excessive heat from the outside of the thermoelectric cooler to the heat sink. In one embodiment, the present invention includes two fans connected in parallel. Alternatively, the fans are connected in series.

In one embodiment, the battery assembly includes a USB C connector and reversible connector diodes for electrostatic discharge (ESD) protection. In yet another embodiment, the headset includes a noise dampening and/or cancelling component that is configured to reduce noise produced by the cooling assemblies.

In one embodiment, the control electronics include software. The software includes a hardware abstraction layer (HAL), a primary abstraction layer (PAL), and an application layer. The HAL includes device drivers and low-level functions that interact with the hardware functions on the microcontroller unit. The PAL includes higher-level firmware functions that include turning the TECs on or off, setting the TECs to desired voltages, printing data to console and converting raw thermistor readings to temperature. The application layer includes functions configured for device behavior such as therapy control and user interaction.

In one embodiment, the headset is further configured to work with a control unit as disclosed in U.S. patent application Ser. No. 17/226,749, which is incorporated herein by reference in its entirety.

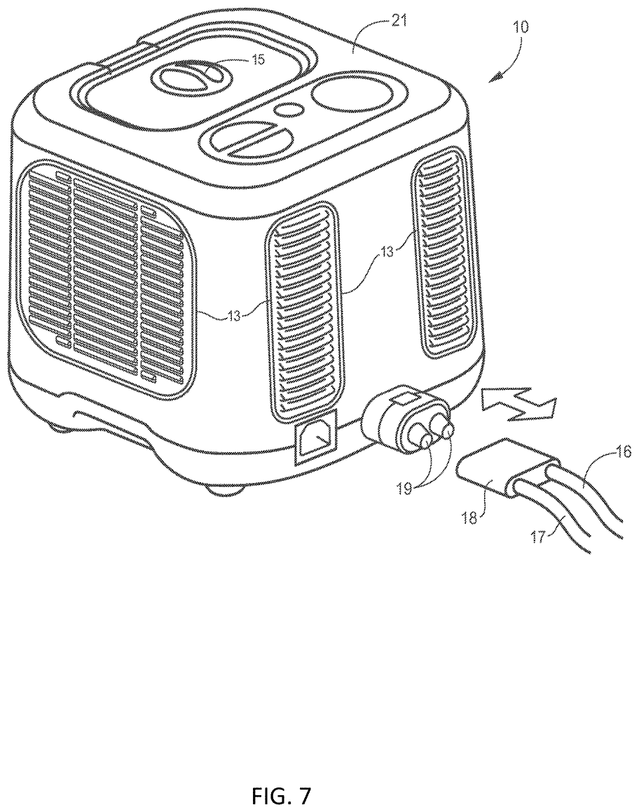

In a preferred embodiment, the virtual reality and augmented reality system includes a headset to change the body temperature of a wearer. In one embodiment, the headset is configured to connect to a control unit. FIG. 7 illustrates a thermoelectric control unit according to one embodiment of the present invention. The control unit 10 is configured to attach through flexible conduit to a temperature-conditioned article, such as the headset. The headset includes internal flexible (e.g., silicon) tubing designed for circulating heated or cooled fluid within a hydraulic circuit between the control unit 10 and the headset. The flexible conduit assembly for the control unit 10 includes separate fluid supply and return lines 16, 17 connected to tubing, and a quick-release female connector 18 for ready attachment and detachment to external male connectors 19 of the control unit 10.

As illustrated in FIG. 7, the exemplary control unit 10 has an external housing 21, and a fluid reservoir located inside the housing 21. The reservoir has a fill opening accessible through a removably capped opening 15 in the housing 21, a fluid outlet, and a fluid return. Fluid contained in the reservoir is moved in a circuit through a conduit assembly formed from in-housing tubes, the flexible supply and return lines 16, 17, and flexible silicone tubing within the temperature-regulated headset. The fluid is selectively cooled, as described further below, by cooperating first and second heat exchangers and thermoelectric cooling modules. The cooling modules reside at an electrified junction between the first and second heat exchangers, and function to regulate fluid temperature from a cool point of as low as 7.78° C. (46° F.), or cooler. In one embodiment, the housing 21 and reservoir are separately or integrally constructed of any suitable material, such as an anti-flammable ABS, polypropylene, or other molded polymer.

From the reservoir, the temperature conditioned fluid exits through the outlet and enters the conduit assembly formed from an arrangement of in-housing Z-, L-, 7-, and S-shaped tubes (and joints). A pump is operatively connected to the reservoir and functions to circulate the fluid through the control unit 10 in a circuit including the in-housing tubes (and joints), flexible fluid supply line 16, silicone pad tubes, fluid return line 17, and back into the reservoir through fluid return. In one embodiment, the control unit includes an insulated linear heat tube located outside of the fluid reservoir and inside the housing. The insulated linear heat tube communicates with the conduit assembly to selectively heat fluid moving from the control unit 10 to the temperature-regulating headset. The exemplary heat tube is operable to heat fluid moving in the hydraulic circuit to a desired temperature as warm as 47.78° C. (118° F.).

The control unit has at least one fluid reservoir. In one embodiment, the control unit includes two fluid reservoirs. A first fluid reservoir is used to heat and/or cool fluid that circulates through the temperature-regulated headset. The first fluid reservoir includes at least one sensor to measure a level of the fluid. A second fluid reservoir is used to store fluid. In a preferred embodiment, fluid from the second fluid reservoir is automatically used to fill the first fluid reservoir when the at least one sensor indicates that the level of the fluid is below a minimum value. Advantageously, this optimizes the temperature in the first fluid reservoir because only a small amount of stored fluid is introduced into the first fluid reservoir when needed. Additionally, this embodiment reduces the refilling required for the control unit, saving the wearer time and effort. In one embodiment, the at least one fluid reservoir is formed of metal. In another embodiment, the metal of the at least one fluid reservoir is electrically connected to ground.

In another embodiment, the control unit includes at least one fluid reservoir holding fluid to be circulated through a temperature-regulated headset. The at least one fluid reservoir is positioned adjacent to at least one thermoelectric module containing at least one Peltier chip. In one embodiment, the at least one thermoelectric module contains four Peltier chips. When electricity is provided to the at least one thermoelectric module, the at least one Peltier chip causes heating or cooling of the at least one fluid reservoir. The at least one thermoelectric module is connected to a first heat sink and a second heat sink via at least one heat tube. In one embodiment, the first heat sink and the second heat sink are aligned along a common axis with space separating the first heat sink from the second heat sink. The at least one thermoelectric module is positioned such that it is not aligned along the common axis of the first heat sink and the second heat sink and such that the at least one thermoelectric module is not located in the space between the first heat sink and the second heat sink. When the at least one thermoelectric module is in cooling mode, at least one heat tube operates to transfer heat away from the at least one thermoelectric module and toward the first heat sink and the second heat sink. This transfer of heat increases the efficiency of the at least one thermoelectric module and prevents the at least one thermoelectric module from breaking down due to excessive heat buildup.

Furthermore, in one embodiment, the control unit includes a partition, separating the common air path from the at least one fluid reservoir. Including the partition helps to ensure that the temperature of the at least one fluid reservoir is not directly affected by the temperature of the air, which decreases the thermal efficiency of the device.

In another embodiment, the thermoelectric module includes at least one first Peltier chip that is separated from at least one second Peltier chip. The at least one first Peltier chip heats and/or cools fluid traveling to a first location, while the at least one second Peltier chip heats and/or cools fluid traveling to a second location, such that the at least one first Peltier chip is part of an independent thermal regulation loop than the at least one second Peltier chip. Separating the at least one first Peltier chip and the at least one second Peltier chip allows the control unit to regulate two different articles at different temperatures. For example and not limitation, the control unit is configured to simultaneously regulate two virtual reality headsets at different temperatures.

For further description about the control unit, see US Patent Publication No. 2018/0110960, which is incorporated herein by reference in its entirety.

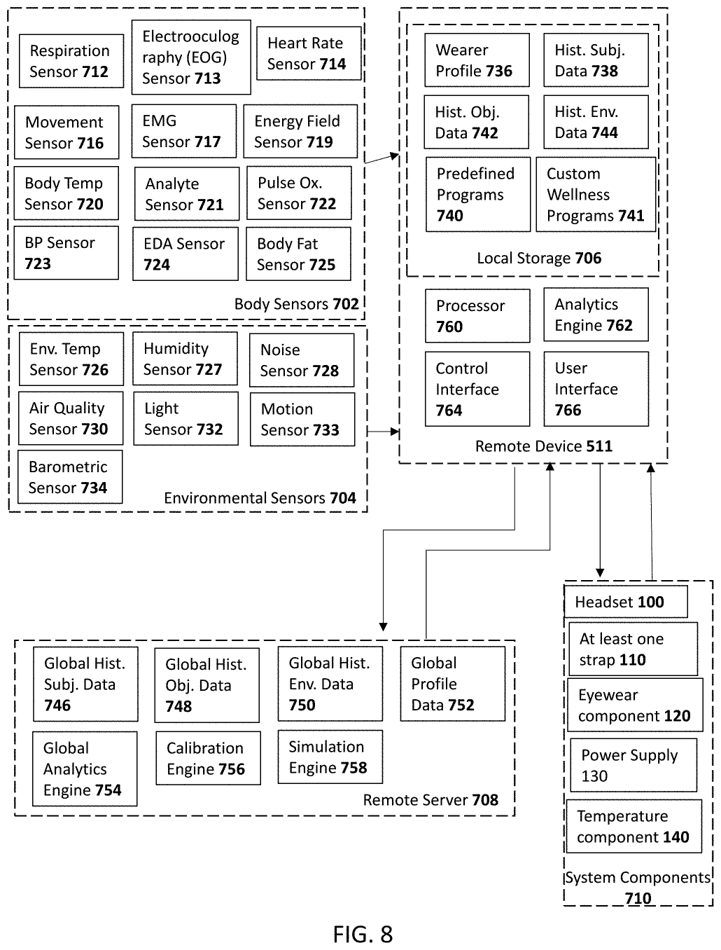

FIG. 8 is a block diagram of one embodiment of the virtual reality system. The virtual reality system includes body sensors 702, environmental sensors 704, a remote device 511 with local storage 706, a remote server 708, and system components 710. The body sensors 702 include a respiration sensor 712, an electrooculography (EOG) sensor 713, a heart rate sensor 714, a movement sensor 716, an electromyography (EMG) sensor 717, a brain wave sensor 718, a body temperature sensor 720, an analyte sensor 721, a pulse oximeter sensor 722, a blood pressure (BP) sensor 723, an electrodermal activity (EDA) sensor 724, and/or a body fat sensor 725.

The respiration sensor 712 measures a respiratory rate. In one embodiment, the respiration sensor 712 is incorporated into the headset. In another embodiment, the respiration sensor 712 is incorporated into a patch or a bandage. Alternatively, the respiratory rate is estimated from an electrocardiogram, a photoplethysmogram (e.g., a pulse oximeter), and/or an accelerometer. In yet another embodiment, the respiratory sensor 712 uses a non-contact motion biomotion sensor to monitor respiration. The respiratory rate enables the virtual reality system to determine how a wearer is responding to the virtual reality environment. The virtual reality system is operable to respond to the wearer's condition by changing the displayed virtual reality imagery and/or the audio or by providing a heating or cooling effect to the wearer.

The electrooculography (EOG) sensor 713 measures the corneo-retinal standing potential that exists between the front and the back of the eye. Measurements of eye movements are done by placing pairs of electrodes either above and below the eye or to the left and right of the eye. If the eye moves to a position away from the center and toward one of the electrodes, a potential difference occurs between the electrodes. The recorded potential is a measure of the eye's position. The eye movement data enables the virtual reality system to determine how the virtual reality environment is impacting a wearer (e.g. motion sickness or restlessness). The virtual reality system is further operable to generate a response based on the eye movement data.

The heart rate sensor 714 is preferably incorporated into the headset. In one embodiment, the heart rate sensor 714 is incorporated into a band that is wrapped around a wearer's head. Alternatively, the heart rate sensor 714 is attached to the wearer with a chest strap. In another embodiment, the heart rate sensor 714 is incorporated into a patch or a bandage. In yet another embodiment, the heart rate sensor is incorporated into a sensor device on or under a mattress (e.g., BEDDIT®, EMFIT® QS™). The heart rate is determined using electrocardiography, pulse oximetry, ballistocardiography, or seismocardiography. In one embodiment, the heart rate sensor 714 measures heart rate variability (HRV). HRV is a measurement of the variation in time intervals between heartbeats. A high HRV measurement is indicative of less stress, while a low HRV measurement is indicative of more stress. Studies have linked abnormalities in HRV to diseases where stress is a factor (e.g., diabetes, depression, congestive heart failure). In one embodiment, a Poincaré plot is generated to display HRV on a device such as a smartphone. The heart rate data indicates whether the meditation and other virtual reality applications are having the desired effect on the wearer. The virtual reality system is configured to generate a response based on the wearer's heart rate if the wearer's heart rate is not at or near the desired rate. For example, and not limitation, in one embodiment, the response generated by the virtual reality system includes a cooling effect when the heart rate of a wearer is above a desired heart rate. The virtual reality system is further operable to provide an audio, visual and/or audiovisual stimulus based on the wearer's heart rate. For example, and not limitation, in one embodiment, the virtual reality system is configured to generate audio and visual stimuli simulating waves crashing on a beach. Alternatively, the virtual reality system is operable is operable to receive input from the wearer for audio and visual stimuli that provides a calming effect on the wearer.

The movement sensor 716 is an accelerometer and/or a gyroscope. In one embodiment, the accelerometer and/or the gyroscope are incorporated into the headset. In another embodiment, the accelerometer and/or the gyroscope are incorporated into a smartphone. In alternative embodiment, the movement sensor 716 is a non-contact sensor. In one embodiment, the movement sensor 716 is at least one piezoelectric sensor. In another embodiment, the movement sensor 716 is a pyroelectric infrared sensor (i.e., a “passive” infrared sensor). In yet another embodiment, the movement sensor 716 is at least one pressure sensor embedded in a mattress or mattress topper.

The brain wave sensor 718 is preferably an electroencephalogram (EEG) with at least one channel. In a preferred embodiment, the EEG has at least two channels. Multiple channels provide higher resolution data. The frequencies in EEG data indicate particular brain states. The brain wave sensor 718 is preferably operable to detect delta, theta, alpha, beta, and gamma frequencies. In another embodiment, the brain wave sensor 718 is operable to identify cognitive and emotion metrics, including focus, stress, excitement, relaxation, interest, and/or engagement. In yet another embodiment, the brain wave sensor 718 is operable to identify cognitive states that reflect the overall level of engagement, attention and focus and/or workload that reflects cognitive processes (e.g., working memory, problem solving, analytical reasoning).

The energy field sensor 719 measures an energy field of a wearer. In one embodiment, the energy field sensor 719 is a gas discharge visualization (GDV) device. Examples of a GDV device are disclosed in U.S. Pat. Nos. 7,869,636 and 8,321,010 and U.S. Publication No. 20100106424, each of which is incorporated herein by reference in its entirety. The GDV device utilizes the Kirlian effect to evaluate an energy field. In a preferred embodiment, the GDV device utilizes a high-intensity electric field (e.g., 1024 Hz, 10 kV, square pulses) input to an object (e.g., human fingertips) on an electrified glass plate. The high-intensity electric field produces a visible gas discharge glow around the object (e.g., fingertip). The visible gas discharge glow is detected by a charge-coupled detector and analyzed by software on a computer. The software characterizes the pattern of light emitted (e.g., brightness, total area, fractality, density). In a preferred embodiment, the software utilizes Mandel's Energy Emission Analysis and the Su-Jok system of acupuncture to create images and representations of body systems. The energy field sensor 719 is preferably operable to measure stress levels, energy levels, and/or a balance between the left and right sides of the body.

The body temperature sensor 720 measures core body temperature and/or skin temperature. The body temperature sensor 720 is a thermistor, an infrared sensor, or thermal flux sensor. In one embodiment, the body temperature sensor 720 is incorporated into the headset. In one embodiment, the body temperature sensor 720 is incorporated into an armband or a wristband. In another embodiment, the body temperature sensor 720 is incorporated into a patch or a bandage. In yet another embodiment, the body temperature sensor 720 is an ingestible core body temperature sensor (e.g., CORTEMP®). The body temperature sensor 720 is preferably wireless.

The analyte sensor 721 monitors levels of an analyte in blood, sweat, or interstitial fluid. In one embodiment, the analyte is an electrolyte, a small molecule (molecular weight<900 Daltons), a protein (e.g., C-reactive protein), and/or a metabolite. In another embodiment, the analyte is glucose, lactate, glutamate, oxygen, sodium, chloride, potassium, calcium, ammonium, copper, magnesium, iron, zinc, creatinine, uric acid, oxalic acid, urea, ethanol, an amino acid, a hormone (e.g., cortisol, melatonin), a steroid, a neurotransmitter, a catecholamine, a cytokine, and/or an interleukin (e.g., IL-6). The analyte sensor 721 is preferably non-invasive. Alternatively, the analyte sensor 721 is minimally invasive or implanted. In one embodiment, the analyte sensor 721 is incorporated into the headset. Alternatively, the analyte sensor 721 is incorporated into a patch or a bandage.

The pulse oximeter sensor 722 monitors oxygen saturation. In one embodiment, the pulse oximeter sensor 722 is worn on a finger, a toe, or an ear. In another embodiment, the pulse oximeter sensor 722 is incorporated into a patch or a bandage. The pulse oximeter sensor 722 is preferably wireless. Alternatively, the pulse oximeter sensor 722 is wired. In one embodiment, the pulse oximeter sensor 722 is connected by a wire to a wrist strap or a strap around a hand. In another embodiment, the pulse oximeter sensor 722 is combined with a heart rate sensor 714. In yet another embodiment, the pulse oximeter sensor 722 uses a camera lens on a smartphone or a tablet.

The blood pressure (BP) sensor 723 is a sphygmomanometer. The sphygmomanometer is preferably wireless. Alternatively, the blood pressure sensor 723 estimates the blood pressure without an inflatable cuff (e.g., SALU™ Pulse+). In one embodiment, the blood pressure sensor 723 is incorporated into a wearable device.

The electrodermal activity sensor 724 measures sympathetic nervous system activity. Electrodermal activity is more likely to have high frequency peak patterns (i.e., “storms”) during deep sleep. In one embodiment, the electrodermal activity sensor 724 is incorporated into a wearable device. Alternatively, the electrodermal activity sensor 724 is incorporated into a patch or a bandage.

The body fat sensor 725 is preferably a bioelectrical impedance device. In one embodiment, the body fat sensor 725 is incorporated into a smart scale (e.g., FITBIT® ARIA®, NOKIA® Body+, GARMIN® INDEX™, UNDER ARMOUR® Scale, PIVOTAL LIVING® Smart Scale, IHEALTH® Core). Alternatively, the body fat sensor 725 is a handheld device.

The environmental sensors 704 include an environmental temperature sensor 726, a humidity sensor 727, a noise sensor 728, an air quality sensor 730, a light sensor 732, a motion sensor 733, and/or a barometric sensor 734. In one embodiment, the environmental temperature sensor 726, the humidity sensor 727, the noise sensor 728, the air quality sensor 730, the light sensor 732, the motion sensor 733, and/or the barometric sensor 734 are incorporated into a home automation system (e.g., AMAZON® ALEXA®, APPLE® HOMEKIT™, GOOGLE® HOME™, IF This Then THAT® (IFTTT®), NEST®). Alternatively, the environmental temperature sensor 726, the humidity sensor 727, the noise sensor 728, and/or the light sensor 732 are incorporated into a smartphone or tablet. In one embodiment, the noise sensor 728 is a microphone. In one embodiment, the air quality sensor 730 measures carbon monoxide, carbon dioxide, nitrogen dioxide, sulfur dioxide, particulates, and/or volatile organic compounds (VOCs).

The remote device 511 is preferably a smartphone or a tablet. Alternatively, the remote device 511 is a laptop or a desktop computer. The remote device 511 includes a processor 760, an analytics engine 762, a control interface 764, and a user interface 766. The remote device 511 accepts data input from the body sensors 702 and/or the environmental sensors 704. The remote device also accepts data input from the remote server 708. The remote device 511 stores data in a local storage 706.

The local storage 706 on the remote device 511 includes a wearer profile 736, historical subjective data 738, predefined programs 740, custom wellness programs 741, historical objective data 742, and historical environmental data 744. The wearer profile 736 stores virtual reality system preferences and information about the wearer, including but not limited to, age, weight, height, gender, medical history (e.g., sleep conditions, medications, diseases), fitness (e.g., fitness level, fitness activities), sleep goals, stress level, and/or occupational information (e.g., occupation, shift information). The medical history includes caffeine consumption, alcohol consumption, tobacco consumption, use of prescription sleep aids and/or other medications, blood pressure, restless leg syndrome, narcolepsy, headaches, heart disease, sleep apnea, depression, stroke, diabetes, insomnia, anxiety or post-traumatic stress disorder (PTSD), and/or neurological disorders.

In one embodiment, the medical history incorporates information gathered from the Epworth Sleepiness Scale (ESS), the Insomnia Severity Index (ISI), Generalized Anxiety Disorder 7-item (GAD-7) Scale, and/or Patient Heath Questionanaire-9 (PHQ-9) (assessment of depression). The ESS is described in Johns M W (1991). “A new method for measuring daytime sleepiness: the Epworth sleepiness scale”, Sleep, 14 (6): 540-5 which is incorporated herein by reference in its entirety. The ISI is described in Morin et al. (2011). “The Insomnia Severity Index: Psychometric Indicators to Detect Insomnia Cases and Evaluate Treatment Response”, Sleep, 34(5): 601-608, which is incorporated herein by reference in its entirety. The GAD-7 is described in Spitzer et al., “A brief measure for assessing generalized anxiety disorder: the GAD-7”, Arch Intern Med., 2006 May 22; 166(1):1092-7, which is incorporated herein by reference in its entirety. The PHQ-9 is described in Kroenke et al., “The PHQ-9: Validity of a Brief Depression Severity Measure”, J. Gen. Intern. Med, 2001 September; 16(9): 606-613, which is incorporated herein by reference in its entirety.

In one embodiment, the weight of the wearer is automatically uploaded to the local storage from a third-party application. In one embodiment, the third-party application obtains the information from a smart scale (e.g., FITBIT® ARIA®, NOKIA® BODY+™, GARMIN® INDEX™, UNDER ARMOUR® Scale, PIVOTAL LIVING® Smart Scale, IHEALTH® Core). In another embodiment, the medical history includes information gathered from a Resting Breath Hold test.

The historical objective data 742 includes information gathered from the body sensors 702. This includes information from the respiration sensor 712, the electrooculography sensor 713, the heart rate sensor 714, the movement sensor 716, the electromyography sensor 717, the brain wave sensor 718, the energy field sensor 719, the body temperature sensor 720, the analyte sensor 721, the pulse oximeter sensor 722, the blood pressure sensor 723, the electrodermal activity sensor 724 and/or the body fat sensor 725. In another embodiment, the historical objective data 742 includes information gathered from the Maintenance of Wakefulness Test, the Digit Symbol Substitution Test, and/or the Psychomotor Vigilance Test. The Maintenance of Wakefulness Test is described in Doghramji, et al., “A normative study of the maintenance of wakefulness test (MWT)”, Electroencephalogr. Clin. Neurophysiol., 1997 November; 103(5): 554-562, which is incorporated herein by reference in its entirety. The Digit Symbol Substitution Test is described in Wechsler, D. (1997). Wechsler Adult Intelligence Scale—Third edition (WAIS-III). San Antonio, Tex.: Psychological Corporation and Wechsler, D. (1997). Wechsler Memory Scale—Third edition (WMS-III). San Antonio, Tex.: Psychological Corporation, each of which is incorporated herein by reference in its entirety. The Psychomotor Vigilance Test is described in Basner et al., “Maximizing sensitivity of the psychomotor vigilance test (PVT) to sleep loss”, Sleep, 2011 May 1; 34(5): 581-91, which is incorporated herein by reference in its entirety.

The historical environmental data 744 includes information gathered from the environmental sensors 704. This includes information from the environmental temperature sensor 726, the humidity sensor 727, the noise sensor 728, the air quality sensor 730, the light sensor 732, and/or the barometric sensor 734.

The historical subjective data 738 includes information regarding sleep and/or stress. In one embodiment, the information regarding sleep is gathered from manual sleep logs (e.g., Pittsburgh Sleep Quality Index). The manual sleep logs include, but are not limited to, a time sleep is first attempted, a time to fall asleep, a time of waking up, hours of sleep, number of awakenings, times of awakenings, length of awakenings, perceived sleep quality, use of medications to assist with sleep, difficulty staying awake and/or concentrating during the day, difficulty with temperature regulation at night (e.g., too hot, too cold), trouble breathing at night (e.g., coughing, snoring), having bad dreams, waking up in the middle of the night or before a desired wake up time, twitching or jerking in the legs while asleep, restlessness while asleep, difficulty sleeping due to pain, and/or needing to use the bathroom in the middle of the night. The Pittsburgh Sleep Quality Index is described in Buysse, et al., “The Pittsburgh sleep quality index: A new instrument for psychiatric practice and research”. Psychiatry Research. 28 (2): 193-213 (May 1989), which is incorporated herein by reference in its entirety.

In another embodiment, the historical subjective data 738 includes information gathered regarding sleepiness (e.g., Karolinska Sleepiness Scale, Stanford Sleepiness Scale, Epworth Sleepiness Scale). The Karolinska Sleepiness Scale is described in Akerstedt, et al., “Subjective and objective sleepiness in the active individual”, Int J Neurosc., 1990; 52:29-37 and Baulk et al., “Driver sleepiness—evaluation of reaction time measurement as a secondary task”, Sleep, 2001; 24(6):695-698, each of which is incorporated herein by reference in its entirety. The Stanford Sleepiness Scale is described in Hoddes E. (1972). “The development and use of the stanford sleepiness scale (SSS)”. Psychophysiology. 9 (150) and Maclean, et al. (1992 Mar. 1) “Psychometric evaluation of the Standford Sleepinees Scale”. Journal of Sleep Research. 1 (1): 35-39, each of which is incorporated herein by reference in its entirety.

In yet another embodiment, the historical subjective data 738 includes information regarding tension or anxiety, depression or dejection, anger or hostility, and/or fatigue or inertia gathered from the Profile of Mood States. The Profile of Mood States is described in the Profile of Mood States, 2^{nd} Edition published by Multi-Health Systems (2012) and Curran et al., “Short Form of the Profile of Mood States (POMS-SF): Psychometric information”, Psychological Assessment 7 (1): 80-83 (1995), each of which is incorporated herein by reference in its entirety. In another embodiment, the historical subjective data 738 includes information gathered from the Ford Insomnia Response to Stress Test (FIRST), which asks how likely a respondent is to have difficulty sleeping in nine different situations. The FIRST is described in Drake et al., “Vulnerability to stress-related sleep disturbance and hyperarousal”, Sleep, 2004; 27:285-91 and Drake et al., “Stress-related sleep disturbance and polysomnographic response to caffeine”, Sleep Med., 2006; 7:567-72, each of which is incorporated herein by reference in its entirety. In still another embodiment, the historical subjective data 738 includes information gathered from the Impact of Events, which assesses the psychological impact of stressful life events. A subscale score is calculated for intrusion, avoidance, and/or hyperarousal. The Impact of Events is described in Weiss, D. S., & Marmar, C. R. (1996). The Impact of Event Scale—Revised. In J. Wilson & T. M. Keane (Eds.), Assessing psychological trauma and PTSD (pp. 399-411). New York: Guilford, which is incorporated herein by reference in its entirety. In one embodiment, the historical subjective data 738 includes information gathered from the Social Readjustment Rating Scale (SRRS). The SRRS lists 52 stressful life events and assigns a point value based on how traumatic the event was determined to be by a sample population. The SRRS is described in Holmes et al., “The Social Readjustment Rating Scale”, J. Psychosom. Res. 11(2): 213-8 (1967), which is incorporated herein by reference in its entirety.

The predefined programs 740 are general virtual reality settings for virtual reality applications. For example, and not limitations, the virtual reality applications include meditation applications, relaxation applications, sleep applications and other similar applications designed to create a physiological effect of the wearer via an immersive virtual environment. Advantageously, the predefined programs 740 are designed for various conditions and/or body types (e.g., weight loss, comfort, athletic recovery, anxiety, depression, alternative sleep cycles). In one embodiment, a meditation program sets a skin temperature at a very cold setting (e.g., 15.56-18.89° C. (60-66° F.)) to decrease brain activity, which then helps a wearer meditate and/or sleep. Temperature settings are automatically adjusted to maximize the immersive environment while having the smallest impact on comfort. Advantageously, the virtual reality system is configured to determine whether a predefined program is having a desired effect on the wearer based on data captured by the body sensors. The virtual reality system is operable to provide temperature, haptic, audio, visual, and/or audiovisual feedback to generate a desired effect on the wearer. For example, and not limitation, if the virtual reality system determines via a temperature sensor or a HRV sensor that a wearer's body temperature is rising or that the wearer is experiencing an elevated heart rate, then the virtual reality system is configured to generate a cooling effect via the control unit and/or the virtual reality headset.

In yet another embodiment, temperature modulation cycles are used to reduce insomnia. One cause of insomnia is the core body temperature failing to drop or a delay of the drop in core body temperature. In one example, the surface temperature is 20° C. (68° F.) at the start of a sleep period, 17.78° C. (64° F.) during N1-N2 sleep, 15.56° C. (60° F.) during N3 sleep, 18.89° C. (66° F.) during REM sleep, and 20° C. (68° F.) to wake the wearer.

In still another embodiment, temperature modulation cycles are used to reduce sleep disruptions due to multiple sclerosis (MS). In MS, core temperature and extremity temperature management are not consistent. As a result, a warm to sleep and warm to wake is suggested. In one example, the surface temperature is 37.78° C. (100° F.) at the start of a sleep period, 21.11° C. (70° F.) during N1-N2 sleep, 20° C. (68° F.) during N3 sleep, 26.67° C. (80° F.) during REM sleep, and 37.78° C. (100° F.) to wake the wearer.

In yet another embodiment, temperature modulation cycles are used to support wearers with alternative sleep cycles. An alternative sleep cycle is when a wearer changes to a multiple phase sleep cycle in a 24-hour cycle (e.g., biphasic, segmented, polyphasic sleep). In one example, the surface temperature is 21.11° C. (70° F.) at the start of a sleep period, 17.78° C. (64° F.) during N1-N2 sleep, 16.67° C. (62° F.) during N3 sleep, 19.44° C. (67° F.) during REM sleep, and 21.11° C. (70° F.) to wake the wearer. This program repeats for multiple, evenly spaced sleep blocks or be used in a longer block of 4-5 hours. For a short 30-minute block, the temperature drops (e.g., 0.278° C./minute (0.5° F./minute) or greater).

The temperature modulation cycles alternate cooling and heating based on automated collection of risk factors, including temperature, surface area pressure, and moisture (e.g., sweat). In another embodiment, temperature modulation cycles are prescribed by a sleep specialist or physician based on a particular health condition of a wearer.

The custom wellness programs 741 are virtual reality settings defined by the wearer. The virtual reality settings include eye sensitivity, motion sickness factors, noise levels, and camera speed settings. In one example, the wearer creates a custom program by modifying a predefined program (e.g., the meditation program above) to be 1.11° C. (2° F.) cooler during the meditation application. In another example, the wearer creates a custom program by modifying a predefined program (e.g., the meditation program) to have a start temperature of 37.78° C. (100° F.). In yet another example, the wearer creates a custom program by modifying the display settings. For example, and not limitation, the display movement, the rate of display movement, and whether any audio should be played is modifiable by the wearer. The custom wellness programs 741 allow a wearer to save preferred virtual reality settings. For example, and not limitation, the custom wellness programs include visual fitness programs, interval training, yoga training, and group training.

Other physical embodiments include a blanket, glove, seat pad, sofa, chair, nap pods, gaming chairs, beds, or the like.

The remote server 708 includes global historical subjective data 746, global historical objective data 748, global historical environmental data 750, global profile data 752, a global analytics engine 754, a calibration engine 756, and a simulation engine 758. The global historical subjective data 746, the global historical objective data 748, the global historical environmental data 750, and the global profile data 752 include data from multiple wearers.

For example, the global analytics engine 754 is operable to suggest settings to wearer based on preferences of other wearers (e.g., similar mental health conditions). The global profile data 752 includes wearer profile data from all wearers. The global historical object data 748 includes historical headset data from all wearers. The global historical environmental data 750 includes historical environmental data from all wearers.

The virtual reality system includes access to external information sources. The external information sources include, but are not limited to, external databases and/or third-party systems. Examples of information stored in external databases includes, but is not limited to, map data, road data, terrain data, and/or meditation data. Examples of third-party systems include, but are not limited to, mobile applications related to wearables (e.g., body sensors) and external motion detection sensors. The external motion sensors collect three-dimensional (3D) data, including, but not limited to, 3D body positional data. The external motion sensors utilize infrared and/or 3D video analysis to obtain the 3D body positional data.

The body sensors 702, the environmental sensors 704, the remote device 511 with local storage 706, the remote server 708, and the system components 710 are designed to connect directly (e.g., Universal Serial Bus (USB) or equivalent) or wirelessly (e.g., BLUETOOTH®, WI-FI®, ZIGBEE®) through systems designed to exchange data between various data collection sources. In a preferred embodiment, the body sensors 702, the environmental sensors 704, the remote device 511 with local storage 706, the remote server 708, and the system components 710 communicate wirelessly through BLUETOOTH®. Advantageously, BLUETOOTH® emits lower electromagnetic fields (EMFs) than WI-FI® and cellular signals.

In a preferred embodiment, the at least one remote device is operable to set target temperatures for the headset. The at least one remote device preferably has a user interface (e.g., a mobile application for a smartphone or tablet, buttons on a remote control) that allows a wearer to select target temperatures for the headset. In one embodiment, the headset includes temperature probes throughout the headset to provide temperature data to the at least one processor, which compares a target temperature set using the at least one remote device to an actual measured temperature to determine whether to heat or cool the wearer.

Those skilled in the art will recognize that programmatic control of the target temperatures over time, such as over the course of a meditation session, is possible using the at least one remote device. Because the target temperatures are able to be set at any time, those target temperatures are able to be manipulated through the meditation session in order to match wearer preferences.

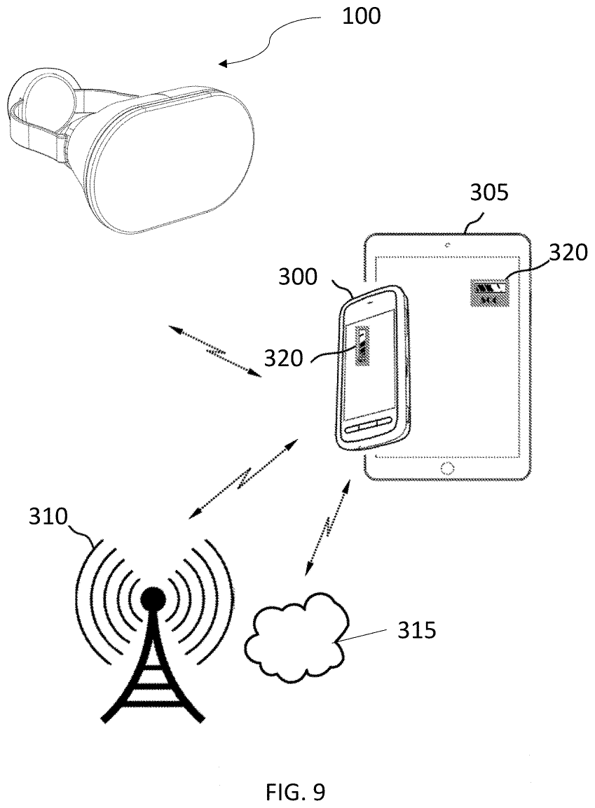

As shown in FIG. 9, the headset 100 is operable to communicate, preferably wirelessly, with at least one remote device, such as but not limited to, a mobile phone 300 or a tablet 305. The mobile phone 300 is able to include any mobile phone that (1) is capable of running mobile applications and (2) is capable of communicating with the headset. In one embodiment, the mobile phone 300 is, for example, an ANDROID™ phone, an APPLE® IPHONE®, or a SAMSUNG® GALAXY® phone. Likewise, the tablet 305 is able to include any tablet that (1) is capable of running mobile applications and (2) is capable of communicating with the headset. The tablet 305 includes, for example, the 3G or 4G version of the APPLE® IPAD® or the 5G version of the Samsung Galaxy Tab S6.

Further in the virtual reality system, the mobile phone 300 and/or the tablet 305 is in communication with a cellular network 310 and/or a network 315. The network 315 is able to be any network for providing wired or wireless connection to the Internet, such as a local area network (LAN) or a wide area network (WAN).

A virtual reality mobile application 320 is installed and running at the mobile phone 300 and/or the tablet 305. The virtual reality system mobile application 320 is implemented according to the type (i.e., the operating system) of mobile phone 300 and/or tablet 305 on which it is running. The virtual reality system mobile application 320 is designed to receive wearer information from the headset 100. Virtual reality mobile application 320 indicates graphically, audibly, and/or tactilely, the physiological condition to the wearer (not shown).

The virtual reality system is operable to utilize a plurality of learning techniques including, but not limited to, machine learning (ML), artificial intelligence (AI), deep learning (DL), neural networks (NNs), artificial neural networks (ANNs), support vector machines (SVMs), Markov decision process (MDP), and/or natural language processing (NLP). The virtual reality system is operable to use any of the aforementioned learning techniques alone or in combination.

Further, the virtual reality system is operable to utilize predictive analytics techniques including, but not limited to, machine learning (ML), artificial intelligence (AI), neural networks (NNs) (e.g., long short term memory (LSTM) neural networks), deep learning, historical data, and/or data mining to make future predictions and/or models. The virtual reality is preferably operable to recommend and/or perform actions based on historical data, external data sources, ML, AI, NNs, and/or other learning techniques. The virtual reality system is operable to utilize predictive modeling and/or optimization algorithms including, but not limited to, heuristic algorithms, particle swarm optimization, genetic algorithms, technical analysis descriptors, combinatorial algorithms, quantum optimization algorithms, iterative methods, deep learning techniques, and/or feature selection techniques.

The virtual reality system is operable to predict how a wearer will respond to a virtual reality environment and apply a heating and/or cooling effect and/or change the display accordingly. Advantageously, the virtual reality system is configured to develop a personalized profile based on a wearer's prior virtual reality environments and response.

In one embodiment, the virtual reality system is in network communication with at least one fluid control unit for at least one thermoregulated article (e.g., a mattress pad, a mattress, a blanket, a pillow, etc.). Exemplary fluid control units and corresponding thermoregulated articles are found at least in U.S. Pat. Nos. 10,278,511 and 11,147,389, and U.S. Patent Publication Nos. 2021/0100378 and 2020/0077942, each of which is incorporated herein by reference in its entirety. In one embodiment, based on a selected virtual environment displayed by the virtual reality system, the virtual reality system transmits messages to the at least one fluid control unit to modulate temperature of the at least one thermoregulated article to match the virtual environment. By way of example and not limitation, if the rendered virtual environment shows the user in the middle of the Savannah, the at least one thermoregulated article increases in temperature to approximate the feeling of being in the Savannah. Contrastingly, if the rendered virtual environment shows the user on a windy beach or on a rainy dock, the at least one thermoregulated article decreases in temperature. In one embodiment, the at least one thermoregulated article is regulated to match the user's precise position in the virtual environment. For example, if the user is in a sunny environment, but under a tree, with only the user's face not in the shade, a pillow is increased in temperature (along with virtual reality system itself), while a mattress pad, mattress, or blanket is kept at a relative normal temperature. By using temperature to match a user's virtual environment, the virtual reality system is better able to immerse the user in the environment, increasing comfort and improving meditation.

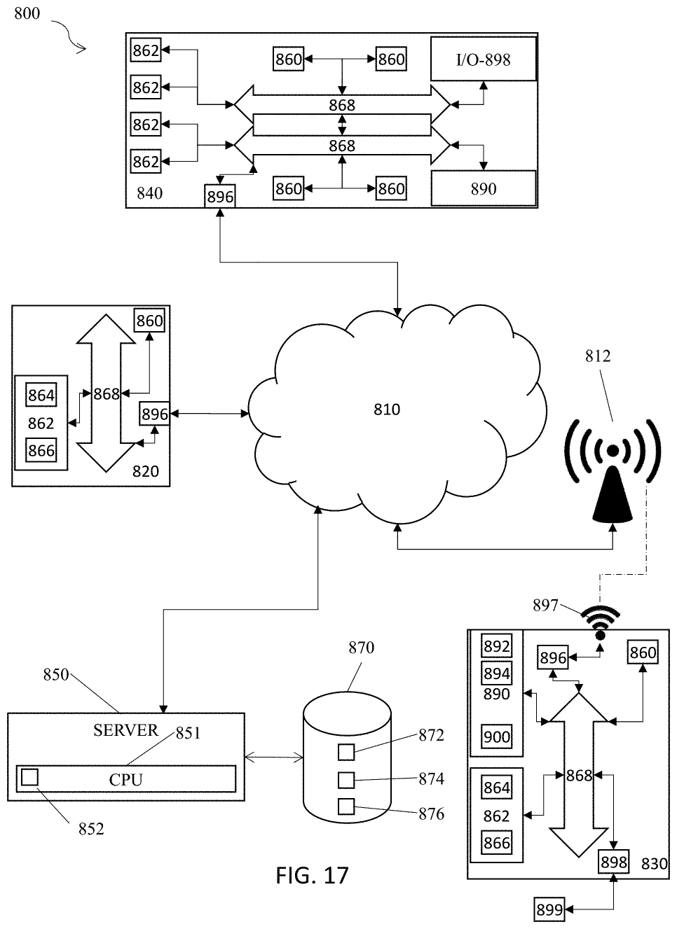

FIG. 17 is a schematic diagram of an embodiment of the invention illustrating a computer system, generally described as 800, having a network 810, a plurality of computing devices 820, 830, 840, a server 850, and a database 870.

The server 850 is constructed, configured, and coupled to enable communication over a network 810 with a plurality of computing devices 820, 830, 840. The server 850 includes a processing unit 851 with an operating system 852. The operating system 852 enables the server 850 to communicate through network 810 with the remote, distributed user devices. Database 870 is operable to house an operating system 872, memory 874, and programs 876.

In one embodiment of the invention, the system 800 includes a network 810 for distributed communication via a wireless communication antenna 812 and processing by at least one mobile communication computing device 830. Alternatively, wireless and wired communication and connectivity between devices and components described herein include wireless network communication such as WI-FI, WORLDWIDE INTEROPERABILITY FOR MICROWAVE ACCESS (WIMAX), Radio Frequency (RF) communication including RF identification (RFID), NEAR FIELD COMMUNICATION (NFC), BLUETOOTH including BLUETOOTH LOW ENERGY (BLE), ZIGBEE, Infrared (IR) communication, cellular communication, satellite communication, Universal Serial Bus (USB), Ethernet communications, communication via fiber-optic cables, coaxial cables, twisted pair cables, and/or any other type of wireless or wired communication. In another embodiment of the invention, the system 800 is a virtualized computing system capable of executing any or all aspects of software and/or application components presented herein on the computing devices 820, 830, 840. In certain aspects, the computer system 800 is operable to be implemented using hardware or a combination of software and hardware, either in a dedicated computing device, or integrated into another entity, or distributed across multiple entities or computing devices.

By way of example, and not limitation, the computing devices 820, 830, 840 are intended to represent various forms of electronic devices including at least a processor and a memory, such as a server, blade server, mainframe, mobile phone, personal digital assistant (PDA), smartphone, desktop computer, netbook computer, tablet computer, workstation, laptop, and other similar computing devices. The components shown here, their connections and relationships, and their functions, are meant to be exemplary only, and are not meant to limit implementations of the invention described and/or claimed in the present application.

In one embodiment, the computing device 820 includes components such as a processor 860, a system memory 862 having a random access memory (RAM) 864 and a read-only memory (ROM) 866, and a system bus 868 that couples the memory 862 to the processor 860. In another embodiment, the computing device 830 is operable to additionally include components such as a storage device 890 for storing the operating system 892 and one or more application programs 894, a network interface unit 896, and/or an input/output controller 898. Each of the components is operable to be coupled to each other through at least one bus 868. The input/output controller 898 is operable to receive and process input from, or provide output to, a number of other devices 899, including, but not limited to, alphanumeric input devices, mice, electronic styluses, display units, touch screens, signal generation devices (e.g., speakers), or printers.

By way of example, and not limitation, the processor 860 is operable to be a general-purpose microprocessor (e.g., a central processing unit (CPU)), a graphics processing unit (GPU), a microcontroller, a Digital Signal Processor (DSP), an Application Specific Integrated Circuit (ASIC), a Field Programmable Gate Array (FPGA), a Programmable Logic Device (PLD), a controller, a state machine, gated or transistor logic, discrete hardware components, or any other suitable entity or combinations thereof that can perform calculations, process instructions for execution, and/or other manipulations of information.

In another implementation, shown as 840 in FIG. 17, multiple processors 860 and/or multiple buses 868 are operable to be used, as appropriate, along with multiple memories 862 of multiple types (e.g., a combination of a DSP and a microprocessor, a plurality of microprocessors, one or more microprocessors in conjunction with a DSP core).

Also, multiple computing devices are operable to be connected, with each device providing portions of the necessary operations (e.g., a server bank, a group of blade servers, or a multi-processor system). Alternatively, some steps or methods are operable to be performed by circuitry that is specific to a given function.

According to various embodiments, the computer system 800 is operable to operate in a networked environment using logical connections to local and/or remote computing devices 820, 830, 840 through a network 810. A computing device 830 is operable to connect to a network 810 through a network interface unit 896 connected to a bus 868. Computing devices are operable to communicate communication media through wired networks, direct-wired connections or wirelessly, such as acoustic, RF, or infrared, through an antenna 897 in communication with the network antenna 812 and the network interface unit 896, which are operable to include digital signal processing circuitry when necessary. The network interface unit 896 is operable to provide for communications under various modes or protocols.

In one or more exemplary aspects, the instructions are operable to be implemented in hardware, software, firmware, or any combinations thereof. A computer readable medium is operable to provide volatile or non-volatile storage for one or more sets of instructions, such as operating systems, data structures, program modules, applications, or other data embodying any one or more of the methodologies or functions described herein. The computer readable medium is operable to include the memory 862, the processor 860, and/or the storage media 890 and is operable be a single medium or multiple media (e.g., a centralized or distributed computer system) that store the one or more sets of instructions 900. Non-transitory computer readable media includes all computer readable media, with the sole exception being a transitory, propagating signal per se. The instructions 900 are further operable to be transmitted or received over the network 810 via the network interface unit 896 as communication media, which is operable to include a modulated data signal such as a carrier wave or other transport mechanism and includes any delivery media. The term “modulated data signal” means a signal that has one or more of its characteristics changed or set in a manner as to encode information in the signal.

Storage devices 890 and memory 862 include, but are not limited to, volatile and non-volatile media such as cache, RAM, ROM, EPROM, EEPROM, FLASH memory, or other solid state memory technology; discs (e.g., digital versatile discs (DVD), HD-DVD, BLU-RAY, compact disc (CD), or CD-ROM) or other optical storage; magnetic cassettes, magnetic tape, magnetic disk storage, floppy disks, or other magnetic storage devices; or any other medium that can be used to store the computer readable instructions and which can be accessed by the computer system 800.

In one embodiment, the computer system 800 is within a cloud-based network. In one embodiment, the server 850 is a designated physical server for distributed computing devices 820, 830, and 840. In one embodiment, the server 850 is a cloud-based server platform. In one embodiment, the cloud-based server platform hosts serverless functions for distributed computing devices 820, 830, and 840.

In another embodiment, the computer system 800 is within an edge computing network. The server 850 is an edge server, and the database 870 is an edge database. The edge server 850 and the edge database 870 are part of an edge computing platform. In one embodiment, the edge server 850 and the edge database 870 are designated to distributed computing devices 820, 830, and 840. In one embodiment, the edge server 850 and the edge database 870 are not designated for distributed computing devices 820, 830, and 840. The distributed computing devices 820, 830, and 840 connect to an edge server in the edge computing network based on proximity, availability, latency, bandwidth, and/or other factors.

It is also contemplated that the computer system 800 is operable to not include all of the components shown in FIG. 17, is operable to include other components that are not explicitly shown in FIG. 17, or is operable to utilize an architecture completely different than that shown in FIG. 17. The various illustrative logical blocks, modules, elements, circuits, and algorithms described in connection with the embodiments disclosed herein are operable to be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application (e.g., arranged in a different order or partitioned in a different way), but such implementation decisions should not be interpreted as causing a departure from the scope of the present invention.

The above-mentioned examples are provided to serve the purpose of clarifying the aspects of the invention, and it will be apparent to one skilled in the art that they do not serve to limit the scope of the invention. By nature, this invention is highly adjustable, customizable and adaptable. The above-mentioned examples are just some of the many configurations that the mentioned components can take on. All modifications and improvements have been deleted herein for the sake of conciseness and readability but are properly within the scope of the present invention.

The invention claimed is:

1. A virtual reality system, comprising:
a virtual reality headset, including a processor and a memory, configured to fit around a user's head and render virtual environments;
at least one headset sensor included in the virtual reality headset and/or at least one wearable sensor in communication with the virtual reality headset;
at least one thermoelectric module included in the virtual reality headset and operable to heat and/or cool a skin-contacting surface of the virtual reality headset; and
wherein the virtual reality headset receives a selection of at least one virtual environment; and
wherein the virtual reality headset transmits instructions to the at least one thermoelectric module to adjust the temperature of the skin-contacting surface of the virtual reality headset based on the selection of the at least one virtual environment and/or sensor data generated by the at least one headset sensor and/or the at least one wearable sensor.

2. The virtual reality system of claim 1, wherein the at least one headset sensor and/or the at least one wearable sensor includes at least one heart rate variability sensor and/or at least one electroencephalography (EEG) sensor.

3. The virtual reality system of claim 1, further comprising at least one fluid control unit, in communication with the virtual reality headset, configured to regulate a temperature of at least one thermoregulating article, wherein the virtual reality headset transmits instructions to the at least one fluid control unit to adjust the temperature of the at least one thermoregulating article based on the selection of the at least one virtual environment and/or the sensor data generated by the at least one headset sensor and/or the at least one wearable sensor.

4. The virtual reality system of claim 3, wherein the at least one thermoregulating article includes at least one mattress pad, at least one mattress, at least one blanket, at least one pillow, at least one piece of furniture, and/or at least one article of clothing.

5. The virtual reality system of claim 1, wherein the at least one virtual environment is automatically changed based on sensor data, generated by the at least one headset sensor and/or the at least one wearable sensor, indicating a stress level of a user.

6. The virtual reality system of claim 1, wherein the selection of the at least one virtual environment is automatically made based on sensor data from the at least one headset sensor embedded included in the virtual reality headset and/or sensor data from the at least one wearable sensor.

7. The virtual reality system of claim 1, wherein the virtual reality headset is in communication with at least one LiDAR sensor, configured to detect changes in a position of the user's body, and wherein the position of an avatar of the user within the at least one virtual reality environment is changed based on sensor data generated by the at least one LiDAR sensor.

8. The virtual reality system of claim 1, wherein the virtual reality headset automatically generates a suggested workout and/or a suggested meditation based on sensor data generated by the at least one headset sensor and/or the at least one wearable sensor.

9. The virtual reality system of claim 1, wherein the virtual reality headset includes a headset control unit, wherein the headset control unit is configured to heat and/or cool fluid, and wherein the fluid exits the headset control unit, enters a thermal regulation medium proximate to the skin-contacting surface, and then reenters the headset control unit.

10. A virtual reality system, comprising:
a virtual reality headset, including a processor and a memory, configured to fit around a user's head and render virtual environments;
at least one headset sensor included in the virtual reality headset and/or at least one wearable sensor in communication with the virtual reality headset; and
a headset control unit, wherein the headset control unit is configured to heat and/or cool fluid;
wherein the fluid exits the headset control unit, enters thermal regulation tubes proximate to a skin-contacting surface of the virtual reality headset, and then reenters the headset control unit;
wherein the virtual reality headset receives a selection of at least one virtual environment; and
wherein the headset control unit heats or cools the fluid based on the at least one selected virtual environment.

11. The virtual reality system of claim 10, wherein the at least one headset sensor includes at least one heart rate variability sensor and/or at least one electroencephalography (EEG) sensor.

12. The virtual reality system of claim 10, wherein the at least one virtual environment is automatically changed based on sensor data, generated by the at least one headset sensor and/or the at least one wearable sensor, indicating a stress level of a user.

13. The virtual reality system of claim 10, wherein the virtual reality headset is in communication with at least one LiDAR sensor, configured to detect changes in a position of the user's body, and wherein the position of an avatar of the user within the at least one virtual reality environment is changed based on sensor data generated by the at least one LiDAR sensor.

14. The virtual reality system of claim 10, wherein the virtual reality headset automatically generates a suggested workout and/or a suggested meditation based on sensor data generated by the at least one headset sensor and/or the at least one wearable sensor.

15. The virtual reality system of claim 10, wherein the headset control unit heats or cools the fluid based on sensor data from the at least one headset sensor included in the virtual reality headset and/or the at least one wearable sensor in communication with the virtual reality headset.

16. The virtual reality system of claim 10, wherein the virtual reality headset receives the selection of the at least one virtual reality environment from a user device in network communication with the virtual reality headset, and wherein the user device includes a smart phone, a tablet, a computer, and/or a smart watch.

17. A virtual reality system, comprising:
a virtual reality headset, including a processor and a memory, configured to fit around a user's head and render virtual environments;
at least one headset sensor included in the virtual reality headset;
at least one thermoelectric module included in the virtual reality headset and operable to heat and/or cool a skin-contacting surface of the virtual reality headset; and
wherein the at least one headset sensor includes at least one heart rate variability sensor and/or at least one electroencephalography (EEG) sensor;
wherein the at least one headset sensor generates sensor data indicating a stress level of a user;
wherein the virtual reality headset receives a selection of at least one virtual environment; and
wherein the at least one virtual environment is automatically changed based on the sensor data, generated by the at least one headset sensor.

18. The virtual reality system of claim 17, wherein the virtual reality headset automatically generates a suggested workout and/or a suggested meditation based on the sensor data generated by the at least one headset sensor.

19. The virtual reality system of claim 17, wherein the virtual reality headset receives the selection of the at least one virtual reality environment from a user device in network communication with the virtual reality headset, and wherein the user device includes a smart phone, a tablet, a computer, and/or a smart watch.

20. The virtual reality system of claim 17, wherein the at least one thermoelectric module heats and/or cools the skin-contacting surface of the virtual reality headset based on the sensor data from the at least one headset sensor.
